(19) United States
(12) Patent Application Publication
(10) Pub. No.: US 2023/0039070 A1
(43) Pub. Date: Feb. 9, 2023

(54) METHOD FOR IDENTIFYING CELL HAVING SPECIFIC BIOLOGICAL CHARACTERISTICS BY CELL CLASSIFICATION PROCESS AND APPLICATION METHOD THEREOF

(71) Applicant: NEXTGEM INC., Tokyo (JP)

(72) Inventors: Masanori MIYANISHI, Tokyo (JP); Masakazu NAKAJIMA, Tokyo (JP); Isao MIYATSUKA, Tokyo (JP); My An Le, Tokyo (JP)

(73) Assignee: NEXTGEM INC., Tokyo (JP)

(21) Appl. No.: 17/789,279

(22) PCT Filed: Jul. 1, 2020
(86) PCT No.: PCT/JP2020/025915

§ 371 Date: Jun. 27, 2022

(30) Foreign Application Priority Data
Dec. 26, 2019 (JP) PCT/JP2019/051270

(51) Int. Cl. C12M 1/34; G16B 99/00; G01N 33/49

(52) U.S. Cl. CPC C12M 1/34; G16B 99/00; G01N 33/49

(57) ABSTRACT

The present invention relates to a method for identifying a cell (group), a method comprising a cell stratifying process utilizing quantitative physical property data, a method for separating a cell (group) utilizing the cell stratifying process, a method for identifying a molecular marker that identifies a cell (group) utilizing the cell stratifying process, a method for culturing a cell (group) utilizing the cell stratifying process, a program for causing a computer to execute a step of identifying a cell (group) utilizing the cell stratifying process, and a system for analyzing, identifying, or separating a cell (group) utilizing the cell stratifying process.

### TECHNICAL FIELD

The present invention relates to a method for identifying a cell (group), a method comprising a cell stratifying process utilizing quantitative physical property data, a method for separating a cell (group) utilizing the cell stratifying process, a method for identifying a molecular marker that identifies a cell (group) utilizing the cell stratifying process, a method for culturing a cell (group) utilizing the cell stratifying process, a program for causing a computer to execute a step of identifying a cell (group) utilizing the cell stratifying process, and a system for analyzing, identifying, or separating a cell (group) utilizing the cell stratifying process.

### BACKGROUND ART

Although cells in a living body basically have identical gene sets and are produced by division of a single cell called fertilized egg cell, their biological functions are variously different. Cells or groups of cells having certain biological properties are distinguished from other cells on the basis of unique biological functions of cells. For example, cells found in tissues in the living body such as bone marrow and muscle are different in morphology and biological function. Stem cells such as hematopoietic stem cells (HSC) are known to have biological functions: self-renewal capacity and multipotency. It is known that, among stem cells having self-renewal capacity and multipotency, some cells maintain the biological function characterizing these stem cells over a long period of time and some stem cells lose the function in a relatively short period of time (Non Patent Literature 1).

Stem cells that maintain self-renewal capacity and multipotency for a long period of time, e.g., over the lifetime of an individual organism, can be mass-cultured using their self-renewal capacity, and can be differentiated into various cells using their multipotency. Stem cells having such properties are considered to be cells that can be a trump card when regenerative medicine is put to practical use. However, all of these stem cells are cells whose abundance in the living body is extremely rare, and cannot be easily identified and isolated from the living body.

From studies of basic biology so far, it is generally considered that, in each tissue of a multicellular organism, there is a differentiation lineage from tissue stem cells to various differentiated cells, and there is a hierarchy of cell differentiation with tissue stem cells as the most upstream. It is understood that the cell differentiation hierarchy is formed and maintained under certain biological rules (FIG. 1). However, it is extremely difficult to estimate the differentiation lineage relationship between various cells separated from the tissue of the individual organism based on information such as cell morphology, and to reconstruct the conceptually understood cell differentiation hierarchy.

Meanwhile, various cells of the living body have unique states in various physical properties such as the expression level of each gene, the intracellular content of the protein expressed from each gene, the type of protein exposed on the cell surface, the state of post-translational modification of the protein, and the cell morphology, and it is possible to identify cells having a certain biological function by a combination of these physical properties. Conventionally, in order to generally identify and purify cells having various biological functions, a method using a fluorescently labeled monoclonal antibody that recognizes a molecule such as a protein expressed on the surface of each cell and a flow cytometer capable of separating cells according to a combination of fluorescent labels of bound antibodies has been mainly used.

However, in such a method, it is necessary to develop an “optimal combination of monoclonal antibodies” through trial and error in order to make it possible to distinguish cells of interest from other cells and purify the cells. Thus, a highly skilled technique has been required for identifying cells with a specific biological function, isolating the cells from other cells, and purifying the cells. Then, due to the number of combinations of monoclonal antibodies to be tested and the like, in general, such a development method requires enormous time and cost. In fact, since the discovery of murine hematopoietic stem cells (HSC) in 1988, it has taken as much as 30 years to discover and identify, among cells having properties of hematopoietic stem cells, particularly long-term hematopoietic stem cells having properties of maintaining self-renewal capacity and multipotency over the lifetime of an individual mouse (Non Patent Literature 2).

### CITATION LIST

### Non Patent Literature

Non Patent Literature 1: Japanese Journal of Transfusion and Cell Therapy, 2006, Vol. 52, No. 5, pp. 583-588 Non Patent Literature 2: nature, 2016, Vol. 530, pp. 223-227

### SUMMARY OF INVENTION

### Technical Problem

In order to identify and isolate rare cells having a biologically important function (e.g., tissue stem cells and cancer stem cells), which belong to a specific hierarchy of the cell differentiation hierarchy, it is desired to develop a novel method capable of achieving higher efficiency and higher speed than conventional development techniques.

### Solution to Problem

Various cells of the living body including hematopoietic cells basically have the same set of genetic information except for some exceptions, but have unique states in various physical properties such as the expression level of each gene, the intracellular content of the protein expressed from each gene, the type of protein exposed on the cell surface, the state of post-translational modification of the protein, and the cell morphology. The present inventors have found that it is possible to stratify a cell population according to a differentiation hierarchy based on data obtained by quantifying these physical properties, and provide a method for analyzing, identifying, or separating cells located in a specific hierarchy of the cell differentiation hierarchy by utilizing a high-precision classification ability model that performs the stratification, and a system using the method.

That is, in one aspect, the present invention provides a method for identifying cells having a specific biological property, the method comprising the steps of: stratifying each cell or cell fraction utilizing similarity of quantitative physical property data of each cell in a sample to obtain a cell fraction arranged spatial structure or a cell arranged spatial structure similar to a cell differentiation lineage; and identifying one or more regions within the cell fraction arranged spatial structure or the cell arranged spatial structure utilizing biological property data of each cell.

Further, in one aspect, the present invention provides a method for isolating cells using quantitative physical property data, the method comprising the steps of: identifying a region in which cells having a specific biological property are located in a cell fraction arranged spatial structure or a cell arranged spatial structure similar to a cell differentiation lineage obtained by stratifying each cell or cell fraction utilizing similarity of quantitative physical property data of each cell in a sample; and obtaining a gating parameter structure for separating the cells having a specific biological property.

Further, in one aspect, the present invention provides a method for identifying a set of candidate markers for identifying cells having a specific biological property, the method comprising a step of identifying one or more regions, utilizing biological property data of each cell, in a cell fraction arranged spatial structure or a cell arranged spatial structure similar to a cell differentiation lineage obtained by stratifying each cell or cell fraction.

Further, in one aspect, the present invention provides a marker for identifying cells having a specific biological property, the marker including: a quantitative physical property selected from CD217, CD191, CD328, CD34, GPR56, CD275, CD185, CD371, CD45RO, CD192, CD44, CD19, Notch 1, CD21, CD182, CD47, CD133, MRGX2, CD117, CD20, CD368, CD3, CD135, CD130, CD180, CD84, CD221, CD276, CD179b, CD85h, CD105, SUSD2, CD162, CD45RA, CD197, CD5, CXCR7, CD161, CD63, CD10, CCRL2, TRA-2-49, CD366, CD28, CD11_{c}, CD106, CD85, CD87, CD11a, CD35, CD46, CD59, Integrin α9β1, CD99, SSEA-5, CD31, CD147, CD226, CD88, CD40, EGFR, CD131, CD58, CD126, SSEA-4, CD127/IL7R, CD194, CD43, Sialyl Lewis X, CD200R, TCR Δ/β, CD123, CD38, MSC, CD268, CD195, CD181, CD69, CD27, CD1b, CD18, KLRG1, CD24, CD97, CD328, CD79b, CD112, CD155, CD326, CD62L, CD45, TCR γ/δ, CD235ab, CD184, CD8a, CD52, CD49f, CD9, CD109, CD279, CD360, CD143, CD45RA, CD4, CD73, CD172a/b, CD154, CD370, Siglec-8, CD85k, CD170, Siglec-9, CD36, CD8ₐ, CD218ₐ, CD304, ROR1, CD1c, CD57, TNAP, CD114, TSLPR, CD278/ICOS, CD116/CSF2RA, CD103, Tim-4, TLT-2, HLA-E, CD317, CD2, CD71, CD269, CD148, HVEM, CD100, CD45RB, CD166, CD61, CD85d, CD298, CD82, DR3, CD164, CD263, CD215, CD132, CD158, MERTK, CD96, CD156c, CD230, CD66a/c/e, CD255, β2-microglobulin, CD26, Lymphotoxin β Receptor, MSC, NPC, CD32, CD81, CD201, CD274, CD48, CD183, CD243, TRA 54, MUC-13, CD111, CD223, CD22, CD179a, CD7, NKp80, CD334, CD199, CD300c, CD120b, CD360, TRA-1-81, CD102, CD11b/ITGAM, CD141, CD207, CD50, CD352, Delta Opioid Receptor, CD89, CD36L1, APCDD1, CD39, CD49ₑ, CD1a, CD49b, CD30, CD324, CD122/IL2RB, CD267, CD90, CD335, CD294, CD196, HLA-A, B, C, CD303, CD200, CD229, CD95, CD213α1, CD13, CD62P, VEGFR-3, CD206, CD140a, TRA-1-60-R, CD92, CD64, Jagged 2, CCR10, CLEC4A, CD107a, FcεRIα, CD355, CD15, CD142, CD272, CD93, CD271, 4-1BB Ligand, CD158b, CD365, Ksp37, IL-28RA, CD85, CD94, CD369, CD74, CD115, CD42b, CD282, CD152, CD340, CD338, CD119, GARP, LOX-1, CD151, CD337, CD29, CD104, CD1d, CD55, GPR83, CXCL16, CD261, CD318, CD25, Notch3, HLA-DR, CD85, TIGIT, CD124, CD6, CD172_{g}, CD245, CD107b, CD258, CD165, CD262, CD336, erbB3, XCR1, CD38, CD138, CD70, CD16, CD144, CD213α2, CD344, CD323, Integrin β7, CD160, CD158e1, CD319, CD49c, CD354, CD314, IFN-γ R b chain, CD23, CD231, CD203c, CD140b, B7-H4, and Notch 4; or a combination of these quantitative physical properties, wherein the cells are long-term hematopoietic stem cells.

Further, in one aspect, the present invention provides a detection kit for identifying cells having a specific biological property,

the detection kit comprising a reagent for quantifying or detecting a quantitative physical property selected from CD217, CD191, CD328, CD34, GPR56, CD275, CD185, CD371, CD45RO, CD192, CD44, CD19, Notch 1, CD21, CD182, CD47, CD133, MRGX2, CD117, CD20, CD368, CD3, CD135, CD130, CD180, CD84, CD221, CD276, CD179b, CD85h, CD105, SUSD2, CD162, CD45RA, CD197, CD5, CXCR7, CD161, CD63, CD10, CCRL2, TRA-2-49, CD366, CD28, CD11c, CD106, CD85, CD87, CD11a, CD35, CD46, CD59, Integrin α9β1, CD99, SSEA-5, CD31, CD147, CD226, CD88, CD40, EGFR, CD131, CD58, CD126, SSEA-4, CD127/IL7R, CD194, CD43, Sialyl Lewis X, CD200R, TCR Δ/β, CD123, CD38, MSC, CD268, CD195, CD181, CD69, CD27, CD1b, CD18, KLRG1, CD24, CD97, CD328, CD79b, CD112, CD155, CD326, CD62L, CD45, TCR γ/δ, CD235ab, CD184, CD8a, CD52, CD49f, CD9, CD109, CD279, CD360, CD143, CD45RA, CD4, CD73, CD172a/b, CD154, CD370, Siglec-8, CD85k, CD170, Siglec-9, CD36, CD8a, CD218ₐ, CD304, ROR1, CD1c, CD57, TNAP, CD114, TSLPR, CD278/ICOS, CD116/CSF2RA, CD103, Tim-4, TLT-2, HLA-E, CD317, CD2, CD71, CD269, CD148, HVEM, CD100, CD45RB, CD166, CD61, CD85d, CD298, CD82, DR3, CD164, CD263, CD215, CD132, CD158, MERTK, CD96, CD156c, CD230, CD66a/c/e, CD255, β2-microglobulin, CD26, Lymphotoxin β Receptor, MSC, NPC, CD32, CD81, CD201, CD274, CD48, CD183, CD243, TRA-2-54, MUC-13, CD111, CD223, CD22, CD179a, CD7, NKp80, CD334, CD199, CD300c, CD120b, CD360, TRA-1-81, CD102, CD11b/ITGAM, CD141, CD207, CD50, CD352, Delta Opioid Receptor, CD89, CD36L1, APCDD1, CD39, CD49e, CD1a, CD49b, CD30, CD324, CD122/IL2RB, CD267, CD90, CD335, CD294, CD196, HLA-A, B, C, CD303, CD200, CD229, CD95, CD213α1, CD13, CD62P, VEGFR-3, CD206, CD140a, TRA-1-60-R, CD92, CD64, Jagged 2, CCR10, CLEC4A, CD107a, FcεRIα, CD355, CD15, CD142, CD272, CD93, CD271, 4-1BB Ligand, CD158b, CD365, Ksp37, IL-28RA, CD85, CD94, CD369, CD74, CD115, CD42b, CD282, CD152, CD340, CD338, CD119, GARP, LOX-1, CD151, CD337, CD29, CD104, CD1d, CD55, GPR83, CXCL16, CD261, CD318, CD25, Notch3, HLA-DR, CD85, TIGIT, CD124, CD6, CD172g, CD245, CD107b, CD258, CD165, CD262, CD336, erbB3, XCR1, CD38, CD138, CD70, CD16, CD144, CD213α2, CD344, CD323, Integrin β7, CD160, CD158e1, CD319, CD49c, CD354, CD314, IFN-γ R b chain, CD23, CD231, CD203c, CD140b, B7-H4, and Notch 4; or a combination of these quantitative physical properties, wherein the cells are long-term hematopoietic stem cells.

Further, in one aspect, the present invention provides a method for culturing cells, the method comprising the steps of: identifying one or more regions, utilizing biological property data of each cell, in a cell fraction arranged spatial structure or a cell arranged spatial structure similar to a cell differentiation lineage obtained by stratifying each cell or cell fraction utilizing similarity of quantitative physical property data of each cell in a sample; and culturing a cell population under conditions where the identified cells are enriched.

Further, in one aspect, the present invention provides a method for evaluating a content of cells having a specific biological property in a test sample, the method comprising a step of comparing a cell fraction arranged spatial structure or a cell arranged spatial structure similar to a cell differentiation lineage obtained by stratifying each cell or cell fraction utilizing similarity of quantitative physical property data of each cell in a reference sample, with a cell fraction arranged spatial structure or a cell arranged spatial structure similar to a cell differentiation lineage obtained by stratifying each cell or cell fraction utilizing similarity of quantitative physical property data of each cell in a test sample.

Further, in one aspect, the present invention provides a method for evaluating a differentiation state of cells in a test sample, the method comprising a step of comparing a cell fraction arranged spatial structure or a cell arranged spatial structure similar to a cell differentiation lineage obtained by stratifying each cell or cell fraction utilizing similarity of quantitative physical property data of each cell in a reference sample, with a cell fraction arranged spatial structure or a cell arranged spatial structure similar to a cell differentiation lineage obtained by stratifying each cell or cell fraction utilizing similarity of quantitative physical property data of each cell in a test sample.

Further, in one aspect, the present invention provides a method for evaluating a differentiation direction of cells in a test sample, the method comprising a step of comparing a cell fraction arranged spatial structure or a cell arranged spatial structure similar to a cell differentiation lineage obtained by stratifying each cell or cell fraction utilizing similarity of quantitative physical property data of each cell in a reference sample, with a cell fraction arranged spatial structure or a cell arranged spatial structure similar to a cell differentiation lineage obtained by stratifying each cell or cell fraction utilizing similarity of quantitative physical property data of each cell in a test sample, under a plurality of conditions.

Further, in one aspect, the present invention provides a method for evaluating a culture method, the method comprising a step of comparing a cell fraction arranged spatial structure or a cell arranged spatial structure similar to a cell differentiation lineage obtained by stratifying each cell or cell fraction utilizing similarity of quantitative physical property data of each cell in a reference sample, with a cell fraction arranged spatial structure or a cell arranged spatial structure similar to a cell differentiation lineage obtained by stratifying each cell or cell fraction utilizing similarity of quantitative physical property data of each cell in a test sample, at each time point before and after culture under specific conditions.

Further, in one aspect, the present invention provides a program for causing a computer to execute the steps of: stratifying each cell or cell fraction utilizing similarity of quantitative physical property data of each cell in a sample to obtain a cell fraction arranged spatial structure or a cell arranged spatial structure similar to a cell differentiation lineage; identifying one or more regions within the cell fraction arranged spatial structure or the cell arranged spatial structure utilizing biological property data of each cell; and identifying a position of cells having a specific biological property in the cell fraction arranged spatial structure or the cell arranged spatial structure.

Further, in one aspect, the present invention provides a program for causing a computer to execute a step of visualizing biological property data obtained by testing cells whose position in a cell fraction arranged spatial structure or a cell arranged spatial structure is identified, the cell fraction arranged spatial structure or the cell arranged spatial structure being similar to a cell differentiation lineage obtained by stratifying each cell or cell fraction utilizing similarity of quantitative physical property data of each cell in a sample.

Further, in one aspect of the present invention, there is provided a system comprising a computer,

in which the computer includes:

(1) an input unit that inputs quantitative physical data and biological property data for each cell;

(2) a process unit that generates a cell fraction arranged spatial structure or a cell arranged spatial structure similar to a cell differentiation lineage by a stratifying process; and

(3) an output unit that outputs the cell fraction arranged spatial structure or the cell arranged spatial structure.

Further, in one aspect of the present invention, there is provided a system comprising a computer,

in which the computer includes:

(1) an input unit that inputs quantitative physical data and biological property data for each cell;

(2) a process unit that generates a cell fraction arranged spatial structure or a cell arranged spatial structure similar to a cell differentiation lineage by a stratifying process; and

(3) an output unit that outputs a combination of physical properties of cells having a specific biological property.

### Advantageous Effects of Invention

In the technique of the present invention, in a cell population that changes on a time series basis, the cell population is arranged (stratified) utilizing quantitative physical property data acquired from the cell population at a specific time point, thereby achieving reproduction of the change on a time series basis. The use of the cell fraction arranged spatial structure or the cell arranged spatial structure (pseudo cell lineage) allows for expression and/or analysis of temporal changes in the differentiation process of each cell. For example, the cell fraction arranged spatial structure and/or the cell arranged spatial structure obtained by stratifying cells is visualized by any method, so that it is possible to support the identification of cells at an initial stage of differentiation, i.e., cells close to stem cells, and the grasping of branched differentiation lineage relationship. In particular, in the case of using quantitative physical property data that can be acquired without affecting the survival of cells, the biological functions of the stratified cells can be further confirmed by experiments. The obtained data regarding the biological functions is combined with the quantitative physical property data, thereby enabling more detailed analysis to be rapidly performed.

According to the method of the present invention, it is possible to identify, for example, tissue stem cells and cancer stem cells at a cost of one several thousandths for a period of one several thousandths, as compared with the conventional method, for example. Further, for example, according to the method of the present invention, it is easy to identify various cell states, and it is possible to perform current and future profiling (such as a health or disease state) of each individual and to provide beneficial information for providing a personalized therapy. Further, the identification of the cell state of the individual and the relation between the medication history and/or the dietary constituent are investigated, so that it possible to support the screening of the personalized therapeutic agent and/or support the personalized instruction of medication and diet. Further, the cell sorting algorithm of the present invention is performed on a cell population obtained after culturing purified cells isolated from a population of cells once obtained from a living body for a certain period of time, thereby allowing for evaluation of the differentiation mode of the purified cells. Furthermore, the relation between the culture conditions when culturing the purified cells for a certain period of time and the evaluation of the differentiation mode is clarified, so that it is possible to support screening for optimal culture conditions for enriching specific differentiating cells from the purified cells.

### BRIEF DESCRIPTION OF DRAWINGS

FIG. 1 is a schematic diagram illustrating a cell differentiation lineage of cells in a biological tissue.

FIG. 2 is a schematic diagram illustrating the similarity between a cell differentiation lineage in a biological tissue and a cell fraction arranged spatial structure or a cell arranged spatial structure generated by a high-precision classification ability model of the present invention.

FIG. 3 is a schematic diagram illustrating an example in which biological property data is applied to the cell fraction arranged spatial structure or the cell arranged spatial structure generated by the high-precision classification ability model of the present invention.

FIG. 4 is a schematic diagram illustrating an example in which biological property data is applied to the cell fraction arranged spatial structure or the cell arranged spatial structure generated by the high-precision classification ability model of the present invention. The drawing shows an example in which a region comprising cells having a biological property of interest is specified from a positional relationship between cells having each of a plurality of biological properties in the cell fraction arranged spatial structure or the cell arranged spatial structure.

FIG. 5 is a schematic diagram illustrating one aspect of a system having a high-precision classification ability model of the present invention.

FIG. 6 is a schematic diagram illustrating one aspect of a process content included in the high-precision classification ability model of the present invention.

FIG. 7 is a schematic diagram illustrating another aspect of the process content included in the high-precision classification ability model of the present invention.

FIG. 8 is a schematic diagram illustrating one aspect of a system having the high-precision classification ability model of the present invention.

FIG. 9 is a schematic diagram illustrating an image displayed on a display of an interface in one aspect of the system having the high-precision classification ability model of the present invention.

FIG. 10 is a schematic diagram 10 illustrating another aspect of the system comprising the high-precision classification ability model of the present invention.

FIG. 11A is a view showing an example of a cell arranged spatial structure of cells contained in human umbilical cord blood, which has been obtained by a method for stratifying cells of the present invention.

FIG. 11B is a view showing an expression level of CD34 in a cell arranged spatial structure of cells contained in human umbilical cord blood, which has been obtained by the method for stratifying cells of the present invention.

FIG. 12A is a view showing an example of a cell arranged spatial structure of a biopsy sample of a head and neck squamous cell carcinoma patient, which has been obtained by the method for stratifying cells of the present invention.

FIG. 12B is a view showing an expression level of P-MET in a cell arranged spatial structure of a biopsy sample of a head and neck squamous cell carcinoma patient, which has been obtained by the method for stratifying cells of the present invention.

FIG. 12C is a view showing predicted distribution of cells which have been confirmed to be cancer cells by biological findings, in a cell arranged spatial structure of a biopsy sample of a head and neck squamous cell carcinoma patient, which has been obtained by the method for stratifying cells of the present invention.

FIG. 12D is a view showing an expression level of ACTA2 in a cell arranged spatial structure of a biopsy sample of a head and neck squamous cell carcinoma patient, which has been obtained by the method for stratifying cells of the present invention.

FIG. 12E is a view showing distribution of cells which have been confirmed to be myofibroblasts by biological findings, in a cell arranged spatial structure of a biopsy sample of a head and neck squamous cell carcinoma patient, which has been obtained by the method for stratifying cells of the present invention.

### DESCRIPTION OF EMBODIMENTS

Hereinafter, embodiments of the present invention will be described. The following descriptions are merely examples, and the scope of the present invention is not limited to these descriptions, and modifications can be appropriately made and implemented without impairing the gist of the present invention.

(Definitions)

In the present specification, when a plurality of ranges of numerical values is indicated, it also means a range comprising a combination of any lower limit value and upper limit value of the plurality of ranges.

The term “differentiation” as used herein refers to a process by which cells having a low degree of specialization are transformed into more specialized cells such as neuronal cells or muscle cells. The term “differentiated” or “differentiating” is a relative term and means that “differentiated cells” or “differentiating cells” are more advanced and specialized in developmental pathway than cells to be compared with the differentiated cells or differentiating cells.

The term “stem cells” as used herein refers to undifferentiated cells that have self-renewal capacity at the single cell level and produce two or more different differentiating cells. Specifically, the stem cell can divide asymmetrically, one daughter cell retains the stem cell state at that time, and the other daughter cell exhibits another specific biological function and phenotype. Alternatively, the stem cells can divide symmetrically into two stem cells, and thus some stem cells are maintained in a cell population having stem cells, whereas other cells in the population only give rise to differentiated progeny.

For example, the term “stem cells” may include pluripotent stem cells, tissue stem cells, and the like. Here, the term “pluripotent” refers to the ability to differentiate into multiple, but limited, cell types. The term “tissue stem cells” include, for example, ectodermal lineage stem cells, mesodermal lineage stem cells, and endodermal lineage stem cells. Whether or not the obtained cells are various stem cells can be determined by the presence or absence of expression of a specific marker gene or the like. The term “stem cells” also includes “cancer stem cells” to be described later.

Also in cancer tissues, it is considered that there are cells having self-renewal capacity and ability (multipotency) to generate cells of various lineages constituting tumors, and such cells are called “cancer stem cells”. “Cancer stem cells” may also be referred to as “tumor initiating cells” or “tumorigenic cells/oncogenic cells”. Similarly to normal tissues, it is understood that there is a differentiation lineage having cancer stem cells as the most upstream in cancer tissues. Therefore, in the present specification, the cells identified utilizing quantitative physical property data also include cells in a cancer tissue, such as “cancer stem cells”.

The term “hematopoietic stem cells” as used herein refers to cells having multipotency over a lifetime that can finally differentiate into all blood cells (red blood cells, white blood cells, platelets, and the like). Hematopoietic stem cells can encompass intermediate stages of differentiation into progenitor cells or blast cells. The term “progenitor cells” or “blast cells” is used interchangeably in the present invention and refers to maturing cells which have a reduced differentiation potential, but are still capable of maturing into different cells of a specific lineage (e.g., bone marrow or lymphoid lineages). In one aspect, the hematopoietic stem cells can be identified by markers Lin⁻, c-Kit⁺, Sca-1⁺, CD150⁺, CD34^{-/low}, and Flk2⁻, or markers Lin⁻, CD48⁻, CD41⁻, and CD150⁺, or the like (Cell, 2005, Vol. 121, pp. 1109-1121).

The term “long-term hematopoietic stem cells” as used herein refers to hematopoietic stem cells that maintain self-renewal capacity for a long time even after undergoing cell division, and at least one of two daughter cells produced by cell division maintains the character of hematopoietic stem cells similar to that of cells before division. In one aspect, the long-term hematopoietic stem cells do not lose any self-renewal capacity by cell division in a normal culture environment in vivo, or in an ex vivo culture environment. Further, in one aspect, the long-term hematopoietic stem cells maintain self-renewal capacity even after undergoing 100, 50, 20 or 10 cell divisions.

The maintenance of the self-renewal capacity can be confirmed by various known methods. For example, the measurement of the content of the undifferentiated cell population contained in the cell group after culturing for a certain period of time makes it possible to confirm that the self-renewal capacity is maintained. The undifferentiated cell population can be identified by, for example, a combination of positivity and negativity for known markers such as c-Kit, Sca-1, and CD11b. Further, in one aspect, the fact that the hematopoietic stem cells maintain self-renewal capacity for a long period of time even after undergoing cell division can be confirmed by a known successive transplantation method.

The term “isolated cells” as used herein refers to cells removed from an organism in which the cells have originated or progeny of such cells. Such cells may have been cultured in vitro, e.g., in the presence of other cells. In addition, such cells or cells that are progeny thereof may be later introduced into the second organism.

The term “isolated cell population” as used herein refers to a population of cells extracted and separated from a heterogeneous cell population. In some aspects, the isolated population may be a substantially pure cell population as compared with the heterogeneous cell population from which the cells have been isolated or enriched, and may still be a heterogeneous cell population.

A specific type of cell population being “substantially pure” means that in the cell population, at least about 50%, more preferably at least about 60%, 65%, 70%, 75%, 85%, 90%, 92%, or 93%, and most preferably at least about 95%, 96%, 97%, 98%, or 99%, of the cells that make up the entire cell population are composed of cells of the specific cell type. For example, the term “substantially pure” stem cell population refers to a population of cells comprising less than about 50%, more preferably less than about 40%, 30%, 20%, 15%, 10%, 8%, 7%, and most preferably less than about 5%, 4%, 3%, 2%, or 1%, of cells that are not stem cells.

The term “identification”, “separation”, “isolation”, “purification”, or “screening” as used herein refers to selecting a target such as an organism, a cell, a substance, or data having a certain property of interest from a population comprising a large number by a specific manipulation/evaluation method. The selected targets may be or need not to be physically separated from the population.

The term “clustering process” or simply “clustering” as used herein refers to data processing that generally divides a set of classification targets into subsets such that internal cohesion and external isolation are achieved (B. S. Everitt: Cluster Analysis, Edward Arnold, third edition (1993), Yasuo Ohashi: A survey of classification techniques, Measurement and control, Vol. 24, No. 11, pp. 999-1006 (1985), Annals of Data Science, 2015, Vol. 2, no. 2, pp 165-193), and is generally classified into “non-hierarchical clustering” and “hierarchical clustering”.

The “non-hierarchical clustering” refers to data processing of defining an evaluation function of the goodness of division and searching for division that optimizes the evaluation function. The non-hierarchical clustering is not particularly limited, and can be performed using a method known to those skilled in the art. For example, the non-hierarchical clustering of the present invention can be performed using a k-means method, a Gaussian mixture model (GMM), or the like, but the method is not limited thereto.

The “hierarchical clustering” means a data process of obtaining a final classification result by sequentially integrating clusters with high similarity based on certain standards while regarding each target as one discrete cluster. The processed result is expressed as a dendrogram or the like that connects all the process targets. The hierarchical clustering analysis is not particularly limited, and can be performed using a method known to those skilled in the art. For example, the hierarchical clustering of the present invention can be performed using the group average method, Ward's method, UPGMA, nearest neighbor method, single linkage method, furthest neighbor method, complete linkage method, or the like, but the method is not limited thereto.

The “quantitative physical property data” as used herein means quantitative data of various physical properties such as the mRNA expression level of each gene, the intracellular content of the protein expressed from each gene, the type of protein exposed on the cell surface, the state of post-translational modification of the protein, and the cell morphology, which are measured for various cells in the living body. Cells having a unique biological function can be specified by a combination of these quantitative physical property data. These physical properties can be quantified based on certain standards according to various known methods, and can be acquired using, for example, a device such as a flow cytometer or a next-generation sequencer (NGS), or a method such as imaging, but there is no limitation thereon. In one aspect, the quantitative physical property data may be obtained by indicating a TRUE/FALSE value showing the presence or absence of physical properties by a pseudo value.

In “stratification” of cells or cell fractions as used herein, a clustering process with the similarity of a target to be processed is performed utilizing the similarity of quantitative physical property data, a positional relationship between each cell cluster (fraction) and/or each cell is defined utilizing the similarity of quantitative physical property data set, and a cell fraction arranged spatial structure and/or a cell arranged spatial structure comprising the positional relationship is obtained.

The term “machine learning” as used herein means that, in a certain aspect, a pattern hidden in data (learning data) is found by a computer according to ordinary understanding of those skilled in the art. Further, it means a method of configuring a classifier that identifies data by learning previously acquired data, and identifying and interpreting newly acquired data by this classifier, in a certain aspect. The learned classifier may be referred to as a “learned model”. An implementation method of machine learning may be selected from artificial neural network learning, decision tree learning, support vector machine learning, Bayesian network learning, clustering, regression analysis, AdaBoost, and the like, but the method is not limited thereto.

The term “flow cytometer” as used herein generally includes three systems: fluidics, optics and electronics, and refers to a known technique of detecting scattered light and fluorescence for individual cells. Information such as the relative size and internal structure of the cell can be obtained from the detected scattered light, and information such as the amounts of various antigens and nucleic acids present in the cell membrane, cytoplasm, and nucleus can be obtained from the fluorescence signal. The scattered light is further classified into two types of forward scattered light (FSC) and side scattered light (SSC) depending on the scattering direction. FSC is light detected in front of the optical axis of the laser beam among scattered light, and its intensity is approximately proportional to the surface area or size of a cell. SSC is light detected at an angle of 90° with respect to the optical axis of the laser beam. Most of the SSC is light scattered when light strikes upon a substance in a cell, and is approximately proportional to the granule shape and internal structure of the cell. Generally, a photodiode is used as a detector for FSC signals, and a highly sensitive photomultiplier tube is used as a detector for SSC and fluorescence signals. Each of the detectors detects an optical signal generated when cells cross the laser beam and generates a voltage pulse proportional to the intensity, so that values of height (H), area (A), and width (W) of the voltage pulse are recorded. These values for FSC and SSC are commonly denoted as FSC—H, FSC-A, FSC—W, SSC—H, SSC-A, SSC—W, and the like. In general, the flow cytometer can simultaneously detect fluorescence in a plurality of wavelength regions. For example, labeling of a plurality of types of cell surface molecules with a plurality of types of fluorescent labels having different emission wavelength regions allows for simultaneous quantification of the amount of these cell surface molecules in each cell. Analysis using the “flow cytometer” technique is called “flow cytometry”.

(Cell Stratifying Process Utilizing Quantitative Physical Property Data)

Various cells of the living body have unique states in various physical properties such as the expression level of each gene, the intracellular content of the protein expressed from each gene, the type of protein exposed on the cell surface, the state of post-translational modification of the protein, and the cell morphology, and it is possible to identify cells having a unique biological function by a combination of these physical properties. These physical properties can be quantified based on certain standards according to various known methods, and thus various cells having a certain biological function can be identified by a combination of the quantitative physical property data.

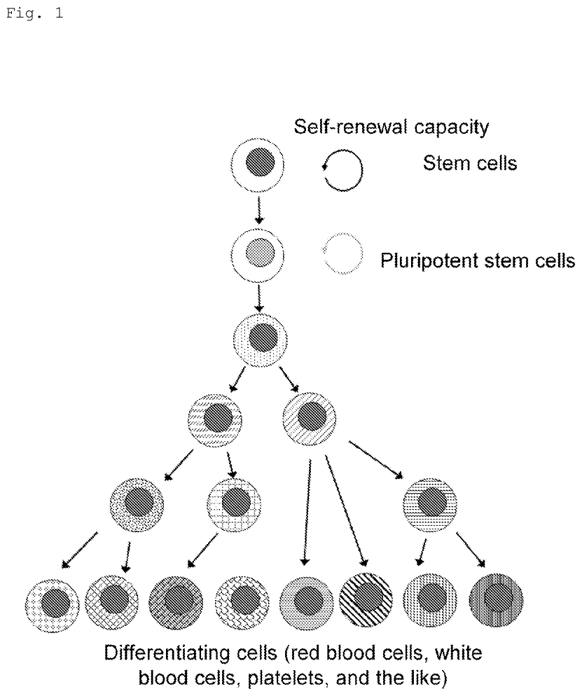

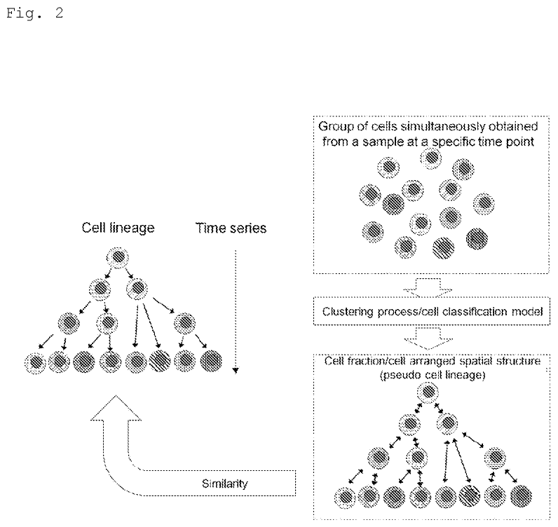

Meanwhile, cell differentiation has a hierarchy (FIG. 1) and it is understood that more closely related cells in the cell differentiation hierarchy are also more similar in their quantitative physical property data. A clustering process with the similarity of a target to be processed is performed utilizing the similarity of quantitative physical property data, a positional relationship between each cell cluster (fraction) and/or each cell is defined utilizing the similarity of quantitative physical property data set, and a cell fraction arranged spatial structure and/or a cell arranged spatial structure comprising the positional relationship is obtained. The cell fraction arranged spatial structure or the cell arranged spatial structure obtained by the clustering process has a structure similar to a cell differentiation lineage tree (FIG. 2).

In the method for identifying cells of the present invention, a cell fraction arranged spatial structure or a cell arranged spatial structure is obtained by cell stratification utilizing physical property data. Typically, in the cell stratification, a positional relationship between each cell or cell fraction is specified utilizing the similarity of quantitative physical property data set by the clustering process utilizing the similarity of each quantitative physical property data, and a cell fraction arranged spatial structure or a cell arranged spatial structure can be obtained as a set of the positional relationships. In one aspect, the cell fraction arranged spatial structure or the cell arranged spatial structure is provided as a data structure in which data representing a positional relationship utilizing the similarity of quantitative physical property data set between cells is accumulated.

In the cell fraction arranged spatial structure or the cell arranged spatial structure, cell fractions and/or cells having similar quantitative physical property data sets occupy close positions. In general, a cell population obtained from a certain amount of biological tissue includes cell groups belonging to various positions on the differentiation lineage. In a case where such a cell group is used as a target to be processed, cell fractions and/or cells at positions close to each other on the differentiation lineage occupy close positions in the cell fraction arranged spatial structure and/or the cell arranged spatial structure obtained by the clustering process. Therefore, the cell fraction arranged spatial structure or the cell arranged spatial structure obtained by the clustering process has a structure similar to the cell differentiation lineage tree.

The cell fraction arranged spatial structure or the cell arranged spatial structure can be expressed as a structure having any number of dimensions depending on the purpose. This dimensional axis is typically expressed as a composite axis of one or more physical properties.

In one aspect, the cell stratifying process of the present invention can be performed by combining biological property data obtained by experiments or the like, in addition to quantitative physical property data. Since cells adjacent to each other in the cell differentiation lineage have similar biological properties, it is possible to obtain a cell fraction arranged spatial structure or a cell arranged spatial structure more similar to the cell differentiation lineage by combining the quantitative physical property data and the biological property data.

(Method for Identifying Cell Group Using Cell Stratifying Process)

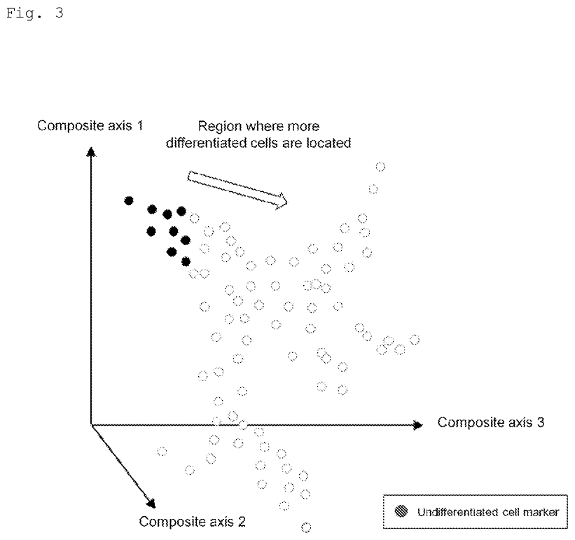

In the cell fraction arranged spatial structure or the cell arranged spatial structure having a structure similar to the cell differentiation lineage tree, the biological property data reflecting the biological function of each cell is given, so that it is possible to identify the position of a cell group having a specific biological property in the cell fraction arranged spatial structure or the cell arranged spatial structure (FIG. 3). For example, in a cell fraction arranged spatial structure or a cell arranged spatial structure composed of a hematopoietic cell group, the position of cells expressing a marker known to be specifically expressed in long-term hematopoietic stem cells which are cells in a more undifferentiated state (the marker is, for example, CD34) is identified, so that it is possible to identify the position of a cell group having biological properties of long-term hematopoietic stem cells in the cell fraction arranged spatial structure or the cell arranged spatial structure.

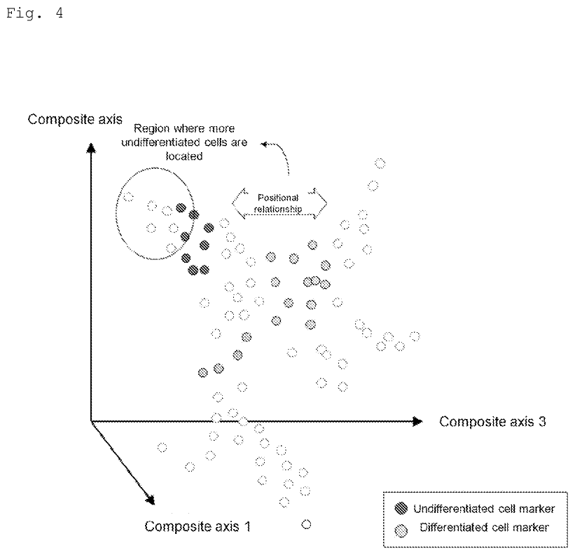

In addition, for example, in a cell fraction arranged spatial structure or a cell arranged spatial structure, the biological property data of each cell is utilized to identify the position of cells belonging to a distant position on the differentiation lineage, so that the relation between the cell fraction arranged spatial structure or the cell arranged spatial structure and the cell differentiation lineage tree can be grasped in detail. For example, a region where cells having more undifferentiated properties on the cell differentiation lineage tree or cells having more differentiated properties are located can be identified in the cell fraction arranged spatial structure or the cell arranged spatial structure (FIG. 4). For such a purpose, for example, markers related to differentiation diversity, functional test data, markers related to self-renewal capacity, and functional test data can be utilized, and a plurality of them can be used in combination.

For example, in the case of being used for hematopoietic cells, the position of cells expressing a marker of long-term hematopoietic stem cells and the position of cells expressing a marker of short-term hematopoietic stem cells are identified, so that it is possible to identify a region where a cell group having properties unique to long-term stem cells is located or a region where a cell group having properties unique to more differentiated cells is located in the cell fraction arranged spatial structure or the cell arranged spatial structure.

More specifically, a region located upstream of the differentiation lineage is identified, so that candidates for long-term hematopoietic stem cells or long-term hematopoietic stem cells can be identified. Long-term hematopoietic stem cells are considered to be a diverse population of cells that include cells differing slightly in various physiological properties, such as self-renewal capacity-maintaining properties and growth properties. In another aspect of the present invention, among the cell populations contained in these long-term hematopoietic stem cells, a subpopulation having particularly similar quantitative physical properties can be identified.

In one aspect, as the biological property data, among properties used for quantitative physical property data, those known to be related to a specific biological property can be used. In addition, experimental results obtained by performing a biological test on an isolated cell group can be used. The test may be an in vitro or in vivo test. The structure of the biological property data is not particularly limited, and may be a scalar value or a TRUE/FALSE value.

In one aspect, the method of combining biological data with the cell fraction arranged spatial structure or the cell arranged spatial structure of the invention of the present application makes it possible to identify a certain biological property having cell group; nothing has been known about the marker to be identified so far, as a combination of quantitative physical property data. Alternatively, a cell group in which some sort of marker is already known is identified with a combination of quantitative physical property data, a more specific marker can be provided. Additionally, a cell group in which a known marker is present can be separated into subpopulations using biological properties.

In one aspect, the clustering process of the present invention includes a step of performing a data clustering process (4) on a quantitative physical property data set obtained by performing a downsampling process (1), a data normalizing process (2), and/or a dimension reducing process (3) on quantitative physical property data. In the downsampling step (1), the amount of data to be subjected to the clustering process is reduced utilizing quantitative physical property data to be analyzed, the distribution of cells, and the like. For this step, for example, a known method such as Density Sampling, SPADE, or Decimate can be applied, but the method is not limited thereto. In the data processing step (2), normalization of quantitative physical property data, unit conversion, and the like are performed using a known method. In the dimension reducing process (3), the dimension of the quantitative physical property data is reduced using a known method. For example, known methods such as PCA, MDS, T-SNE, UMAP, PHATE, and Modified Locally Linear Embedding can be used, but the method is not limited thereto.

The quantitative physical property data of target cells is not particularly limited, and various data obtained by a known method can be utilized. For example, it is possible to acquire quantitative data regarding physical properties such as the intracellular content of mRNA or protein expressed from each gene, the type of protein exposed on the cell surface, the state of post-translational modification of the protein, and the cell morphology, and these properties can be used as quantitative physical property data. In a specific aspect, a flow cytometer can be used for the quantitative physical property data, and various antibodies and reporter proteins can also be utilized. In addition, data obtained by exhaustively quantifying mRNA expressed by each cell using a next generation sequencer (NGS) may be used.

Any number of quantitative physical property data may be used, such as, but not limited to, 1, 2, 3, 4, 5, 6, 7, 8, 9, 10, 11, 12, 13, 14, 15, 16, 17, 18, 19, 20, 25, 30, 40, 50, 60, 70, 80.90, 100, 150, 200, 250, 300, 350, 400, 450, 500, 600, 700, 800, 900, 1000, 2000, 3000, 4000, 5000, 6000, 7000, 8000, 9000, 10000, or more than 10000 quantitative physical property data.

The classification ability in the clustering process depends on a combination of quantitative physical property data sets determined for various cells, setting parameters of the clustering process, and the like. Usually, the type of quantitative physical property data is appropriately set, as a result of which a target cell population can be classified into some forms. For example, the combination of the quantitative physical property data is only required to have a variation in the value to the extent that individual cells can be distinguished in the target cell population. Usually, a sufficient variation can be secured by increasing the type of the quantitative physical property data. At this time, it is preferable to combine quantitative physical property data having greatly different physical properties such as cell morphology and protein expression level, and it is also desirable to combine the expression levels of proteins having different biological functions as quantitative physical property data even in the protein expression level. Further, such variations in the quantitative physical property data can be quantitatively evaluated using a value such as a variance, a standard deviation or a coefficient of variation, and the combination of the quantitative physical property data used in the invention of the present application can be obtained, for example, by combining two or more pieces of property data in which the value of “variance”, “standard deviation”, or “coefficient of variation” between cells is equal to or more than a certain value in the target cell population. Specific numerical values of these values can be appropriately set by those skilled in the art on the basis of a target cell population, a device to be used, accuracy of intended cell classification, and the like. For example, and without limitation, quantitative physical property data having a coefficient of variation of 0.01, 0.02, 0.03, 0.04, 0.05, 0.06, 0.07, 0.08, 0.09, 0.10, 0.15, 0.2, 0.3, 0.4, 0.5, or the like may be used in combination.

(Optimization of Cell Stratifying Process)

The cell stratifying process used in the method for identifying cells utilizing the quantitative physical property data of the present invention depends on the type of the clustering algorithm to be used, and also depends on, for example, setting parameters at the time of performing clustering the number of clusters, or the like.

In one aspect of the present invention, biological properties obtained for cells to be processed can be obtained or determined by experiments or the like, and the cell stratifying process can be optimized utilizing the obtained or determined biological properties. For example, the clustering process can be optimized such that cells having a specific biological property in data of the property are stratified into an identical or neighboring region on the cell fraction arranged spatial structure or the cell arranged spatial structure.

In one aspect, the present invention optimizes the cell stratifying process in order to identify long-term hematopoietic stem cells. Through previous studies, marker molecules possessed by certain long-term hematopoietic stem cells are known. Such marker molecules are used as part of the quantitative physical properties, the clustering process can be optimized such that cell groups showing similarity to data of the properties are stratified into an identical or neighboring region on the cell fraction arranged spatial structure or the cell arranged spatial structure. In such optimization, it is possible to optimize a plurality of quantitative physical properties.

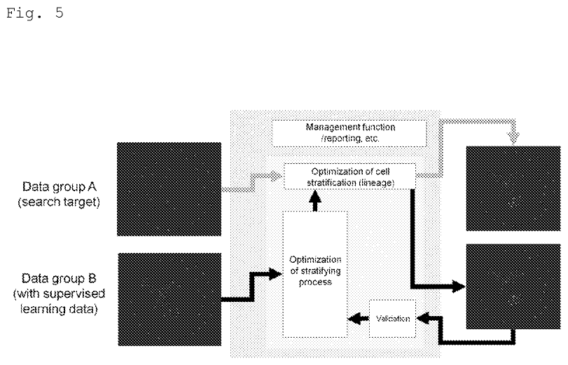

In one aspect of the present invention, separately from the clustering process, a classifier that classifies known stem cells in a tissue to be examined utilizing the above-described quantitative physical property data is configured using a general machine learning method. Then, an analysis is conducted on the classifier (learned model) learned so as to be able to discriminate stem cells in this manner, thereby identifying the degree of importance in the classification of each quantitative physical property data used for classification, and identifying one or more pieces of quantitative physical property data important for discriminating the stem cells as quantitative physical property data for differentiation lineage identification. As described above, the clustering process is optimized using one or more pieces of quantitative physical property data for differentiation lineage identification, specified by the learned model learned using known stem cells, so that it is possible to configure the clustering process of more accurately sorting (stratifying) target cells according to the cell differentiation lineage (FIG. 5).

It is possible to optimize the cell stratifying process used in the method for identifying cells utilizing the quantitative physical property data of the present invention by changing the setting parameters of the clustering process. For example, the optimization can be performed by changing the setting parameters of the downsampling process (1), the data processing process (2), and/or the dimension reducing process (3), which are combined with the clustering process, but the method is not limited thereto.

(Method for Acquiring Quantitative Physical Property Data)

The quantitative physical property data of target cells used in the present invention can be acquired by various known methods capable of quantifying the physical property data of each cell or cell fraction. For example, as the method, a method of quantifying the cell morphology and the expression level of protein on the cell surface using the flow cytometer, a method of quantifying the whole genome mRNA expression level at a single cell level using a device such as a next-generation sequencer (NGS), a method of quantifying various quantitative physical properties by imaging in combination with labeling, or the like can be used, but the method is not limited thereto. In the case of simultaneously quantifying a plurality of cell surface marker molecules, a set of commercially available markers (surface Marker Screening Panel/BD Biosciences, LEGEND SCREENING/BIO LEGEND, and the like) can be used.

In one aspect of the present invention, in a cell population that changes on a time series basis, the cell population is arranged (stratified) utilizing quantitative physical property data acquired from the cell population at a specific time point, thereby reproducing the change on a time series basis. In another aspect, in order to secure the number of cells and/or the diversity of cells, a cell fraction arranged space or a cell arranged space may be generated using quantitative physical property data of a cell population obtained from an analyte at a plurality of time points. Further, quantitative physical property data of a cell population obtained from similar tissues of different analytes may be used in a mixed manner. Accordingly, the quantitative physical property data of the present invention may be data from a cell population obtained from a single analyte or multiple analytes at a plurality of time points.

(Method for Separating Cells Utilizing Cell Stratification)

In one aspect of the present invention, it is possible to separate cells using a cell sorter utilizing a combination of quantitative physical property data that identifies a partial region of a cell fraction arranged spatial structure or a cell arranged spatial structure obtained by cell stratification. The cell sorter can generally separate cells having a specific value range for various physical properties by the gating process. By combining the gating step using a plurality of physical properties used as references, cells or cell populations having a specific value range can be separated for a plurality of quantitative physical properties. The combination of the value ranges of the physical property data used for such separation constitutes a gating parameter structure. For example, long-term hematopoietic stem cells or subpopulations thereof can be separated from a cell population comprising a variety of cells, such as a biological sample, by using a gating parameter structure that reflects a combination of quantitative physical property data for identifying a spatial region comprising the long-term hematopoietic stem cells or subpopulations thereof in the cell fraction arranged spatial structure or the cell arranged spatial structure.

(Method for Selecting Marker for Identifying Cells Utilizing Cell Stratification)

In one aspect of the present invention, a combination of quantitative physical property data that identifies a partial region of a cell fraction arranged spatial structure or a cell arranged spatial structure obtained by cell stratification or a part thereof can be used as a set of markers for identifying cells located in the region, for example, for the purpose of clarifying the localization of the cells in a biological tissue, in addition to the use of cell separation.

In one aspect of the present invention, a set of markers utilized to identify a region in which cells having a specific biological property are located in a cell fraction arranged spatial structure or a cell arranged spatial structure can be used as a set of candidate markers for identifying the cells having the biological property. The biological property of the cells identified by the set of candidate markers is confirmed in more detail, so that it is possible to identify the set of markers necessary for identifying cells having a biological property of interest with desired accuracy. The cell fraction arranged spatial structure or the cell arranged spatial structure of the present invention is utilized, so that a set of candidate markers can be obtained in an extremely short period of time as compared with the case of identifying a set of candidate markers utilizing only biological properties.

In one aspect of the present invention, as a candidate marker for identifying long-term hematopoietic stem cell candidate cells, there is provided a candidate marker including one or more properties selected from CD217, CD191, CD328, CD34, GPR56, CD275, CD185, CD371, CD45RO, CD192, CD44, CD19, Notch 1, CD21, CD182, CD47, CD133, MRGX2, CD117, CD20, CD368, CD3, CD135, CD130, CD180, CD84, CD221, CD276, CD179b, CD85h, CD105, SUSD2, CD162, CD45RA, CD197, CD5, CXCR7, CD161, CD63, CD10, CCRL2, TRA-2-49, CD366, CD28, CD11_{c}, CD106, CD85, CD87, CD11a, CD35, CD46, CD59, Integrin α9β1, CD99, SSEA-5, CD31, CD147, CD226, CD88, CD40, EGFR, CD131, CD58, CD126, SSEA-4, CD127/IL7R, CD194, CD43, Sialyl Lewis X, CD200R, TCR Δ/β, CD123, CD38, MSC, CD268, CD195, CD181, CD69, CD27, CD1b, CD18, KLRG1, CD24, CD97, CD328, CD79b, CD112, CD155, CD326, CD62L, CD45, TCR γ/δ, CD235ₐb, CD184, CD8a, CD52, CD49f, CD9, CD109, CD279, CD360, CD143, CD45RA, CD4, CD73, CD172a/b, CD154, CD370, Siglec-8, CD85k, CD170, Siglec-9, CD36, CD8a, CD218a, CD304, ROR1, CD1_{c}, CD57, TNAP, CD114, TSLPR, CD278/ICOS, CD116/CSF2RA, CD103, Tim-4, TLT-2, HLA-E, CD317, CD2, CD71, CD269, CD148, HVEM, CD100, CD45RB, CD166, CD61, CD85d, CD298, CD82, DR3, CD164, CD263, CD215, CD132, CD158, MERTK, CD96, CD156c, CD230, CD66a/c/e, CD255, β2-microglobulin, CD26, Lymphotoxin β Receptor, MSC, NPC, CD32, CD81, CD201, CD274, CD48, CD183, CD243, TRA-2-54, MUC-13, CD111, CD223, CD22, CD179ₐ, CD7, NKp80, CD334, CD199, CD300c, CD120b, CD360, TRA-1-81, CD102, CD11b/ITGAM, CD141, CD207, CD50, CD352, Delta Opioid Receptor, CD89, CD36L1, APCDD1, CD39, CD49e, CD1a, CD49b, CD30, CD324, CD122/IL2RB, CD267, CD90, CD335, CD294, CD196, HLA-A, B, C, CD303, CD200, CD229, CD95, CD213α1, CD13, CD62P, VEGFR-3, CD206, CD140a, TRA-1-60-R, CD92, CD64, Jagged 2, CCR10, CLEC4A, CD107a, FcεRIα, CD355, CD15, CD142, CD272, CD93, CD271, 4-1BB Ligand, CD158b, CD365, Ksp37, IL-28RA, CD85, CD94, CD369, CD74, CD115, CD42b, CD282, CD152, CD340, CD338, CD119, GARP, LOX-1, CD151, CD337, CD29, CD104, CD1d, CD55, GPR83, CXCL16, CD261, CD318, CD25, Notch3, HLA-DR, CD85, TIGIT, CD124, CD6, CD172_{g}, CD245, CD107b, CD258, CD165, CD262, CD336, erbB3, XCR1, CD38, CD138, CD70, CD16, CD144, CD213α2, CD344, CD323, Integrin β7, CD160, CD158e1, CD319, CD49c, CD354, CD314, IFN-γ R b chain, CD23, CD231, CD203c, CD140b, B7-H4, and Notch 4.

In one aspect, a marker for identifying cells utilizing the physical property data can be used in combination with other known markers or markers for identifying biological properties.

(Detection Kit for Detecting Cells)

When cell surface molecules or the like are selected as a marker for identification utilizing the quantitative physical property data of the present invention, it is possible to provide a detection kit comprising a combination of reagents for chemically detecting and quantifying these molecules. As for such reagents, a labeled antibody having a cell surface molecule as an antigen, a biocompatible polymer having specific affinity for a cell surface molecule, an enzyme protein whose activity is changed by a reaction with a cell surface molecule, or the like can be used as a reagent.

In one aspect of the present invention, as a detection kit for identifying candidate cells as long-term hematopoietic stem cells, there is provided a kit comprising a reagent that reacts with one or more properties selected from CD217, CD191, CD328, CD34, GPR56, CD275, CD185, CD371, CD45RO, CD192, CD44, CD19, Notch 1, CD21, CD182, CD47, CD133, MRGX2, CD117, CD20, CD368, CD3, CD135, CD130, CD180, CD84, CD221, CD276, CD179b, CD85h, CD105, SUSD2, CD162, CD45RA, CD197, CD5, CXCR7, CD161, CD63, CD10, CCRL2, TRA-2-49, CD366, CD28, CD11c, CD106, CD85, CD87, CD11a, CD35, CD46, CD59, Integrin α9β1, CD99, SSEA-5, CD31, CD147, CD226, CD88, CD40, EGFR, CD131, CD58, CD126, SSEA-4, CD127/IL7R, CD194, CD43, Sialyl Lewis X, CD200R, TCR α/β, CD123, CD38, MSC, CD268, CD195, CD181, CD69, CD27, CD1b, CD18, KLRG1, CD24, CD97, CD328, CD79b, CD112, CD155, CD326, CD62L, CD45, TCR γ/δ, CD235ₐb, CD184, CD8a, CD52, CD49f, CD9, CD109, CD279, CD360, CD143, CD45RA, CD4, CD73, CD172a/b, CD154, CD370, Siglec-8, CD85k, CD170, Siglec-9, CD36, CD8a, CD218a, CD304, ROR1, CD1c, CD57, TNAP, CD114, TSLPR, CD278/ICOS, CD116/CSF2RA, CD103, Tim-4, TLT-2, HLA-E, CD317, CD2, CD71, CD269, CD148, HVEM, CD100, CD45RB, CD166, CD61, CD85d, CD298, CD82, DR3, CD164, CD263, CD215, CD132, CD158, MERTK, CD96, CD156c, CD230, CD66a/c/e, CD255, β2-microglobulin, CD26, Lymphotoxin β Receptor, MSC, NPC, CD32, CD81, CD201, CD274, CD48, CD183, CD243, TRA-2-54, MUC-13, CD111, CD223, CD22, CD179a, CD7, NKp80, CD334, CD199, CD300c, CD120b, CD360, TRA-1-81, CD102, CD11b/ITGAM, CD141, CD207, CD50, CD352, Delta Opioid Receptor, CD89, CD36L1, APCDD1, CD39, CD49e, CD1a, CD49b, CD30, CD324, CD122/IL2RB, CD267, CD90, CD335, CD294, CD196, HLA-A, B, C, CD303, CD200, CD229, CD95, CD213α1, CD13, CD62P, VEGFR-3, CD206, CD140a, TRA-1-60-R, CD92, CD64, Jagged 2, CCR10, CLEC4A, CD107a, FcεRIα, CD355, CD15, CD142, CD272, CD93, CD271, 4-1BB Ligand, CD158b, CD365, Ksp37, IL-28RA, CD85, CD94, CD369, CD74, CD115, CD42b, CD282, CD152, CD340, CD338, CD119, GARP, LOX-1, CD151, CD337, CD29, CD104, CD1d, CD55, GPR83, CXCL16, CD261, CD318, CD25, Notch3, HLA-DR, CD85, TIGIT, CD124, CD6, CD172_{g}, CD245, CD107b, CD258, CD165, CD262, CD336, erbB3, XCR1, CD38, CD138, CD70, CD16, CD144, CD213α2, CD344, CD323, Integrin β7, CD160, CD158e1, CD319, CD49c, CD354, CD314, IFN-γ R b chain, CD23, CD231, CD203c, CD140b, B7-H4, and Notch 4.

(Method for Culturing Cells Utilizing Cell Stratification)

The cell stratification of the present invention is utilized to make it possible to optimize culture conditions so that cells having a specific biological property are enriched. For example, a comparison between a cell fraction arranged spatial structure or a cell arranged spatial structure of a cell population at the start of culture and a cell fraction arranged spatial structure or a cell arranged spatial structure of a cell population after culturing for a certain period of time under specific culture conditions allows for determining whether or not the culture conditions lead to enrichment of the cell population having a biological property of interest, and identifying culture conditions that lead to further enrichment. Such optimized culture conditions are utilized, so that cell populations having a specific biological property can be produced rapidly and in large quantities.

(Method for Determining Content of Cells Having Specific Biological Property in Sample Utilizing Cell Stratification)

The cell stratification of the present invention can be utilized to evaluate the content of cells having a specific biological property in a sample. For example, a cell fraction arranged spatial structure or a cell arranged spatial structure obtained by stratifying cells contained in a reference sample is compared with a cell fraction arranged spatial structure or a cell arranged spatial structure obtained by stratifying cells in a test sample, so that it is possible to evaluate the content of cells having a specific biological property in the test sample in comparison with the reference sample. The content of cells having a specific biological property in the sample is evaluated in this manner, so that it is possible to perform, for example, the quality control of cell products. For example, utilization of the present method makes it possible to predict how much long-term hematopoietic stem cells remain after thawing the frozen umbilical cord blood or the like, and to predict how much long-term hematopoietic stem cells are contained in fresh umbilical cord blood or the like.

(Method for Evaluating Differentiation State of Cells Utilizing Cell Stratification)

Utilization of the cell stratification of the present invention makes it possible to identify the differentiation state of cells in a test sample. Typically, a cell fraction arranged spatial structure or a cell arranged spatial structure obtained by stratifying cells in a reference sample is compared with a cell fraction arranged spatial structure or a cell arranged spatial structure obtained by stratifying cells in a test sample, and the deflection of the spatial structure of the test sample with respect to the reference sample is analyzed, thereby allowing for evaluation of the differentiation state of the cell population in the test sample.

(Method for Evaluating Differentiation Direction of Cells Utilizing Cell Stratification)

Utilization of the cell stratification of the present invention makes it possible to evaluate the differentiation direction of cells in a test sample. Typically, a cell fraction arranged spatial structure or a cell arranged spatial structure obtained by stratifying cells contained in a reference sample is compared with a cell fraction arranged spatial structure or a cell arranged spatial structure obtained at a plurality of time points for a part in a test sample, so that it is possible to evaluate which direction the cells in the test sample are differentiating.

(Method for Adjusting Cell Culture Conditions Utilizing Cell Stratification)

The cell stratification of the present invention is utilized to adjust cell culture conditions for the purpose of, for example, enriching for cells having a specific biological property. Typically, the relation of culture conditions between a cell fraction arranged spatial structure or a cell arranged spatial structure obtained by stratifying cells in a reference sample and a cell fraction arranged spatial structure or a cell arranged spatial structure obtained by stratifying cells at each time point before and after culture of a test sample is statistically analyzed, so that it is possible to evaluate culture conditions that give a desired effect, for example, culture conditions that enrich cells having a specific biological property, and to adjust the culture method.

(High-Precision Classification Ability Model and Program for Identifying Cells Utilizing Cell Stratification)

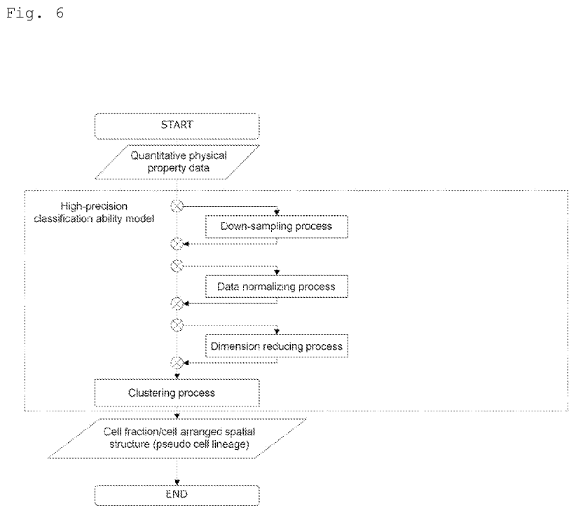

The cell stratifying step used in the method for identifying cells utilizing the quantitative physical property data of the present invention can be used as a processing system that causes a computer to perform a calculation process of sorting a target cell group into cell differentiation hierarchies and providing a cell fraction arranged spatial structure or a cell arranged spatial structure, in combination with the downsampling process (1), the data processing process (2) and/or the dimension reducing process (3) as necessary. The processing system used for the cell stratifying process using the computer is referred to as a “high-precision classification ability model” (FIG. 6). The model is configured as a program for instructing the computer to perform a process, and can be stored in a storage medium readable from the computer.

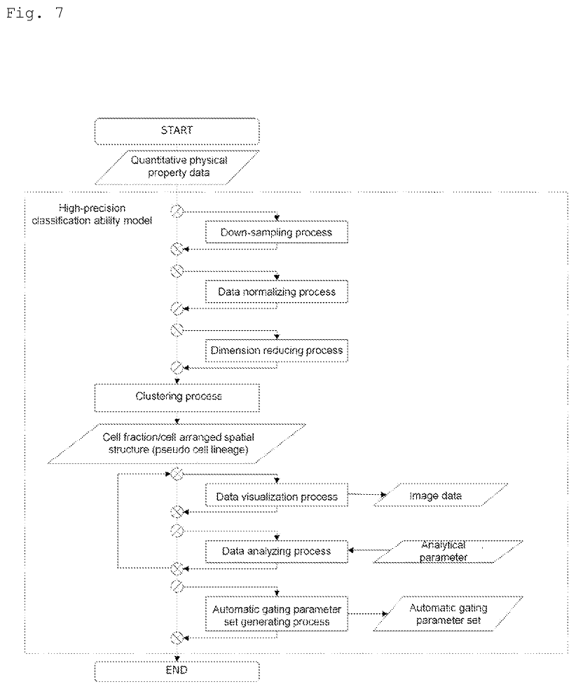

In one aspect, after the clustering (stratifying) process (4) is performed, optionally in combination with the downsampling process (1), the data processing process (2), and/or the dimension reducing process (3), the high-precision classification ability model of the present invention may include a data visualization process (5) that generates image data for visualizing and representing a cell fraction arranged spatial structure or a cell arranged spatial structure obtained by the high-precision classification ability model, an analyzing process (6) that analyzes and/or modifies the cell fraction arranged spatial structure or the cell arranged spatial structure based on input analytical parameters, and an automatic gating parameter set generating process (7) that generates an automatic gating parameter set comprising a combination of quantitative physical property data (quantitative physical property data set) for determining each cell fraction or each cell included in a partial region of the cell fraction arranged spatial structure and/or the cell arranged spatial structure (FIG. 7). For the data visualization process, a known method can be used. For example, Minimum Spanning Tree, ElPiGraph, or the like can be used, but the method is not limited thereto. In addition, a known method can be used for generating the automatic gating parameter set. For example, flowDensity or the like can be used, but the method is not limited thereto. In one aspect, the program of the present invention causes a computer to execute a step for visualizing the cell fraction arranged spatial structure or the cell arranged spatial structure on the display in the form of a pseudo-three-dimensional space or the like.

In one aspect, instead of the data visualization process (5) that generates image data for visualizing and representing a cell fraction arranged spatial structure or a cell arranged spatial structure obtained by the high-precision classification ability model, and the analyzing process (6) that analyzes and/or modifies the cell fraction arranged spatial structure or the cell arranged spatial structure based on input analytical parameters, there may be performed an analyzing process that identifies a region on the cell fraction arranged spatial structure or the cell arranged spatial structure according to a predetermined standard, and analyzes and/or modifies the cell fraction arranged spatial structure or the cell arranged spatial structure.

As the step of identifying a region on the cell fraction arranged spatial structure or the cell arranged spatial structure according to a predetermined standard, for example, a step of selecting a region having specific biological property data, a step of selecting a region satisfying the conditions specified from a positional relationship among a plurality of regions having specific biological property data, and the like can be adopted, but the step is not limited thereto.

As a more specific example, in a case where a hematopoietic cell population is stratified, it is possible to select a region on a cell fraction arranged spatial structure or a cell arranged spatial structure comprising cells having a marker specifically expressed in long-term hematopoietic stem cells, or it is possible to select a region considered to include more undifferentiated cells utilizing a positional relationship between a region comprising cells expressing a marker specifically expressed in long-term hematopoietic stem cells and a region comprising cells expressing a marker specifically expressed in short-term hematopoietic stem cells.

(Analysis Method and Program Utilizing Cell Fraction Arranged Spatial Structure or Cell Arranged Spatial Structure)

In one aspect, the present invention provides an analysis method comprising visualizing biological property data regarding cells in a cell fraction arranged spatial structure or a cell arranged spatial structure similar to a cell differentiation lineage obtained by stratifying cells and evaluating biological properties of cells located in each region of the space, and a program for causing a computer to execute the analysis method.

In one aspect, the present invention provides an analysis method comprising: visualizing biological property data in the spatial structure again, the biological property data being obtained by separating cells whose position in the cell fraction arranged spatial structure or the cell arranged spatial structure is identified, and testing the cells; and evaluating biological properties of cells located in each region of the space, and a program for causing a computer to execute the analysis method.

In one aspect, the present invention provides a method for isolating cells whose position in the cell fraction arranged spatial structure or the cell arranged spatial structure is identified, combining biological property data obtained by testing the cells with physical property data, and newly generating a cell fraction arranged spatial structure or a cell arranged spatial structure utilizing these data, and a program for causing a computer to execute the method.

In one aspect, the present invention provides a method for performing more detailed analysis by visualizing biological property data in the newly generated cell fraction arranged spatial structure or cell arranged spatial structure, and a program for causing a computer to execute the method.

Utilization of these methods and programs allows for integrative and reflexive analysis of biological properties obtained by the step of identifying cells having a desired biological property utilizing data, the biological experiment of the identified cells, and the like. Consequently, cells having a specific biological function can be identified very rapidly.

In one aspect, the program of the present invention can output statistically processed data instead of visualization on the cell fraction arranged spatial structure or the cell arranged spatial structure. The program of the present invention can further execute a data analyzing step based on the output data and the set threshold value.

(System for Identifying Cells Utilizing Cell Stratification)

In one aspect, the present invention provides a system comprising a computer,

in which the computer includes:

(1) an input unit that inputs quantitative physical data and biological property data for each cell;

(2) a process unit that generates a cell fraction arranged spatial structure or a cell arranged spatial structure similar to a cell differentiation lineage by a stratifying process; and

(3) an output unit that outputs the cell fraction arranged spatial structure or the cell arranged spatial structure.

Such a system can be used to correlate biological property data with cell differentiation lineage for analysis.

In one aspect, the present invention provides a system comprising a computer,

in which the computer includes:

(1) an input unit that inputs quantitative physical data and biological property data for each cell;

(2) a process unit that generates a cell fraction arranged spatial structure or a cell arranged spatial structure similar to a cell differentiation lineage by a stratifying process; and

(3) an output unit that outputs a combination of physical properties of cells having a specific biological property.

Such a system can be used to identify cells having a specific biological property.

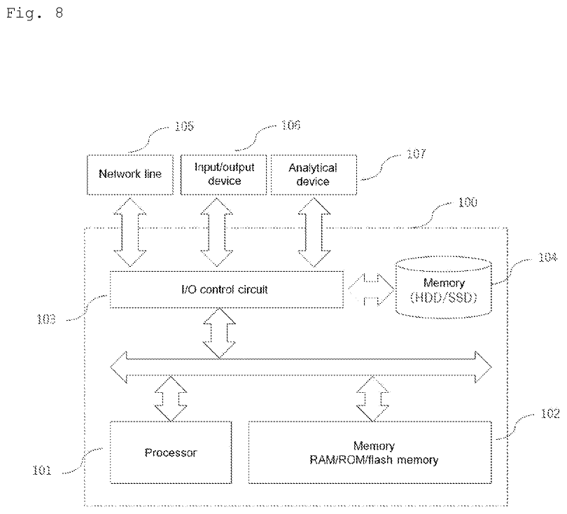

As a block diagram in FIG. 8, there is illustrated a configuration of a computer used in a system that has the high-precision classification ability model of the present invention and processes the model. In this regard, FIG. 8 is merely an example. The computer used in the system having the high-precision classification ability model of the present invention does not need to include all the components illustrated in FIG. 8, and a configuration in which a part is omitted can be adopted, or another component can be added.

A computer 100 illustrated in FIG. 8 includes a processor 101 comprising a CPU or the like, a memory 102 comprising a RAM, a ROM, and/or a flash memory, an I/O control circuit 103, and a main storage device 104 comprising an HDD, an SSD, or the like. These components are electrically connected to each other via a control line and a data line. The I/O control circuit 103 is further connected in a manner similar to a network line 105 outside the computer, an input/output device 106 such as a keyboard, a mouse or a display, and an analytical device 107 such as a flow cytometer or a cell sorter.

In the computer 100 illustrated in FIG. 8, a program code defining each information processing process performed in the high-precision classification ability model is stored in the main storage device 104. The processor 101 reads and executes the program code through the I/O control circuit 103. The memory 102 is temporarily used to write and read data during execution of the program.

The processor 101 can include a single or a plurality of CPUs.

The network circuit connected to the I/O control circuit 103 is a wired or wireless network, and is used for transmission and reception of data necessary for execution of the program, and the like. The input/output device 106 connected to the I/O control circuit 103 includes an input device that is used when a human operates the computer in executing the program, such as a mouse or a keyboard, and/or an output device that causes the human to recognize a result processed by the computer, such as an LCD display or a speaker. The analytical device 107 connected to the I/O control circuit 103 includes a device such as a flow cytometer or a cell sorter, and is used for acquisition of quantitative physical property data or cell isolation.

In one aspect of the present invention, the input/output device 106 includes an image display that displays display image data generated by the high-precision classification ability model.

In one aspect of the present invention, the input/output device 106 includes a process input unit that specifies a partial region on the image displayed on the image display and a process to be performed on the partial region, and transmits the information identifying the selected region and the process to the computer. The computer identifies a region of the cell fraction arranged spatial structure or cell arranged spatial structure data corresponding to the selected region by the data analyzing process of the high-precision classification ability model, and performs the specified process on the spatial structure data.

In one aspect of the present invention, the display image data for displaying the processed result generated by the high-precision classification ability model is displayed on the image display of the input/output device. The process of the analysis and the process of displaying can be repeated any number of times, and a bidirectional human interface can be configured.

The interface can include, for example, an image display that displays, as a two-dimensional image, a graph (image) in which cells included in each cell fraction fractionated by the high-precision classification ability model are plotted in different colors for each fraction in a two-dimensional or three-dimensional space. Further, the interface can include an input unit that can control a viewpoint to be observed. In a case where a graph plotted in a three-dimensional space is displayed as a two-dimensional image, the interface may include an input unit that changes a viewpoint by rotating each axis (dimensional axis) of the three-dimensional space. As such an input unit, a unit which displays a scroll bar, a virtual trackball, a virtual joystick, or the like on the display and in which the scroll bar, the virtual trackball, the virtual joystick, or the like is operated with a mouse pointer or the like displayed on the display may be provided, or the operation of hardware such as a mouse, a track pad or a joystick may be associated with a change in viewpoint.

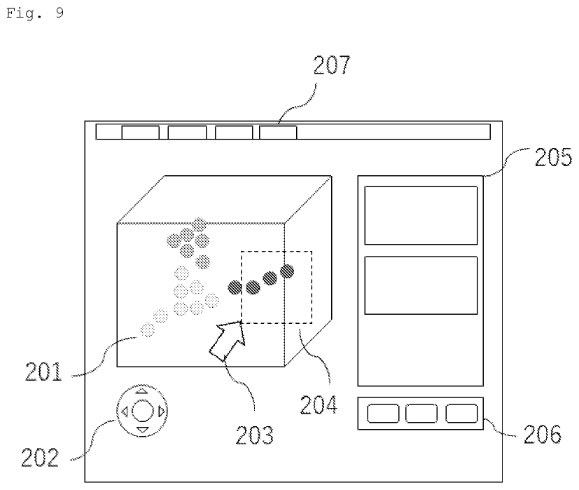

In one aspect of the present invention, an image displayed on the image display of the interface is illustrated in FIG. 9. The image includes a graph (201) in which cells contained in each cell fraction fractionated by the high-precision classification ability model are plotted in different colors for each fraction in a three-dimensional space. The graph can be rotated by operating a virtual joystick (202) with a mouse pointer (203). Further, a region (204) in the graph (201) is specified with the mouse pointer (203), so that it is possible to identify cells or cell fractions included in the region. An information display (205) can display numerical data on all cells used for analysis, data on cells belonging to the region specified in the graph, and the like. Further, a button (206) displayed in the image or a pull-down menu (207) is operated with the mouse pointer, so that it is possible to specify various processes such as analysis of the entire analysis data or cells belonging to the specified region, and storage of data. The drawing is an example, and the present invention may include an interface having another configuration.

In one aspect of the present invention, the analytical device 107 includes a measurement unit for detecting quantitative physical property data from a sample to be analyzed, such as a flow cytometer, a next generation sequencer (NGS), an imaging device or a physical property analyzer. In another aspect, the analytical device 107 includes a sample processing unit for isolating a cell population using a quantitative physical property data set, such as a cell sorter.

In one aspect of the present invention, the measurement unit acquires a plurality of types of quantitative physical property data for each cell to be analyzed, and provides the plurality of types of quantitative physical property data to a computer. The computer can directly process the quantitative physical property data with the high-precision classification ability model, and store all or part of the quantitative physical property data obtained from a plurality of cells contained in the sample to be analyzed in the main storage device as an arbitrarily formatted data structure.

In one aspect of the present invention, the sample processing unit can divide a sample cell group or isolate part of the sample cell group using the quantitative physical property data set generated by the automatic gating parameter set generating process of the high-precision classification ability model of the computer. The cell group obtained from the sample may be the cell group itself used for generating the cell fraction arranged spatial structure or the cell arranged spatial structure data in the high-precision classification ability model, or may be a cell group obtained from a sample of the same type as the sample.

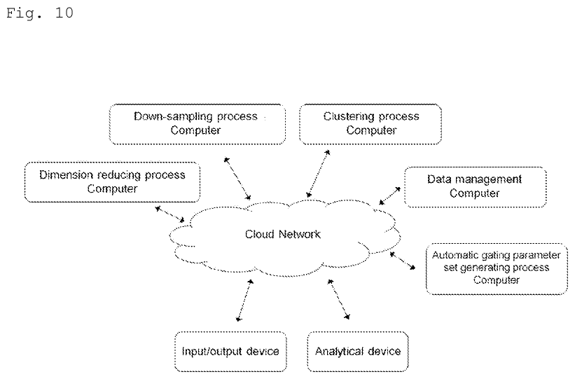

In one aspect of the present invention, a plurality of the computers may be used, and each of the computers may individually perform each process of the high-precision classification ability model and exchange the processed result through a network line. In addition, the input/output device and the analytical device may be connected to the computers via the network line, or the entire system may have a cloud network configuration (FIG. 10).

As a system having the high-precision classification ability model of the present invention, a system comprising a learning unit that generates and extends the high-precision classification ability model and an identifying unit that classifies target cells using the learned high-precision classification ability model can be configured.

If necessary, the system may include other functions such as a management function and a report function used to investigate the state of the high-precision classification ability model (FIG. 5).

Hereinafter, the present invention will be described in more detail with reference to Examples, but the present invention is not limited thereto.

### EXAMPLES

### Example 1: Identification of Long-Term Hematopoietic Stem Cells in Mice

Collection of Cells

Bone marrow cells were collected from the bone marrow of long-term hematopoietic stem cell-specific reporter mice (mice in which the Hoxb5 gene on the genome was replaced with a Hoxb5 gene fused with a gene encoding three copies of mCherry fluorescent protein; Nature, 2016, Vol. 530, pp. 223-227).

Acquirement of Quantitative Physical Data

Quantitative data on 16 physical properties below was acquired.

Specifically, bone marrow cells are suspended in an antibody staining buffer (PBS/2% FCS/2 mM EDTA, etc.), and all the cells are stained on ice. After staining with an anti-c-kit antibody, c-Kit-positive cells were concentrated using Magnetic-activated cell sorting (MACS). Thereafter, the remaining antibodies were sequentially added to the concentrated c-Kit-positive cells for staining, and SYTOX-Red was finally added as a DNA staining reagent for removing dead cells. The stained cells were subjected to data collection using a flow cytometer and dedicated software BD FACSDiva. The machine learning data was analyzed and extracted using FlowJo.

| Parameter | Property name | Content |
|---|---|---|
| Att1 | FSC-A | Forward scattered light (pulse area) |
| Att2 | FSC-H | Forward scattered light (pulse height) |
| Att3 | FSC-W | Forward scattered light (pulse width) |
| Att4 | SSC-A | Side scattered light (pulse area) |
| Att5 | SSC-H | Side scattered light (pulse height) |
| Att6 | SSC-W | Side scattered light (pulse width) |
| Att7 | Fcgr + | Teg receptor or IL-7 receptor-positivity |
| | i17r | |
| Att8 | Sytox- | Sytox-Red staining |
| | Red | |
| Att9 | cKit | cKit protein positivity |
| Att10 | Sca-1 | Sca-1 protein positivity |
| Att11 | Lineage | Lineage marker positivity |
| Att12 | Slamf1 | Slamf1 protein positivity |
| Att13 | CD11b | CD11b protein positivity |
| Att14 | CD34 | CD34 protein positivity |
| Att15 | Flk2 | Flk2 protein positivity |
| Att16 | mCherry | Hoxb5 positivity |

Cell Sorting (Stratifying) Process

Quantitative physical property data for differentiation lineage identification was identified by analyzing a classifier for identifying HSC cell groups based on the obtained quantitative physical property data, thereby optimizing the non-hierarchical clustering process using a Gausian Mixture Model.

Specifically, the process was performed in the following two steps.

(Step 1) Identification of Quantitative Physical Property Data for Differentiation Lineage Identification

First, a machine learning model (AdaBoost) capable of classifying HSCs was constructed from 220,610 pieces of sample data under supervised learning by machine learning using an HSC cell group already identified based on Hoxb5 positivity as correct data. At that time, among the 16 parameters described above, parameters significantly contributing to classification were identified, and it was found that Att12, 9, and 14 were parameters significantly contributing to classification. Thus, Att12, 9 and 14 were identified as quantitative physical property data for differentiation lineage identification. The result of identifying Hoxb5-positive HSC by a combination of these parameters was unexpected.

(Step 2) Clustering Optimization and Clustering

Next, for all the 16 parameters, non-hierarchical clustering (Gausian Mixture Model) was performed without using correct data. At this time, the clustering process was optimized by setting the number of clusters so that the number of clusters comprising Att12, 9, and 14 was minimized. As a result, the clustering process was optimized so as to divide 220,610 pieces of sample data (each sample data represents an individual cell) into 101 clusters. When the sample data included in each of the clusters obtained from the clustering result was confirmed, the clusters comprising cells having Att12, 9, and 14 were 3 out of 101 clusters. 1,198 pieces of sample data in total were classified into the 3 clusters.

As a result of analyzing the classification result, it was confirmed that all the mCherry positive cells, i.e., Hoxb5-positive HSC cells were classified into the 3 clusters, and the clustering process was optimized so that the cells can be stratified according to the differentiation hierarchy of the cell differentiation lineage.

Rapid Identification of Long-term Hematopoietic Stem Cells by Cell Stratification

Concerning total bone marrow nucleated cells (250,304 cases), quantitative physical property data on the above 16 physical properties was acquired, and the result of performing the cell sorting (stratifying) process based on the quantitative physical property data was arranged in a three-dimensional spatial structure and imaged.

Next, the positions where an LT-HSC cell group already identified based on Hoxb5 positivity (331 cases) and an ST-HSC cell group already identified based on Hoxb5 negativity (1,081 cases) were placed in the above three-dimensional spatial structure were validated.

As a result, it was confirmed that the LT-HSC cell group (331 cases) identified based on Hoxb5 positivity and the ST-HSC cell group (1,081 cases) identified based on Hoxb5 negativity were intensively arranged in a specific region in the three-dimensional spatial structure, and arranged according to the order of biological cell differentiation. Specifically, it was confirmed that the LT-HSC cell group and the ST-HSC cell group were arranged in this order starting from one end of the three-dimensional spatial structure.

In addition, the contribution degree of each of the 16 parameters in the arrangement of the cells in the three-dimensional spatial structure was attempted to be quantified, and it was found that Att12, 16, and 10 were parameters significantly contributing to classification.

### Example 2: Stratification of Human Hematopoietic Cells

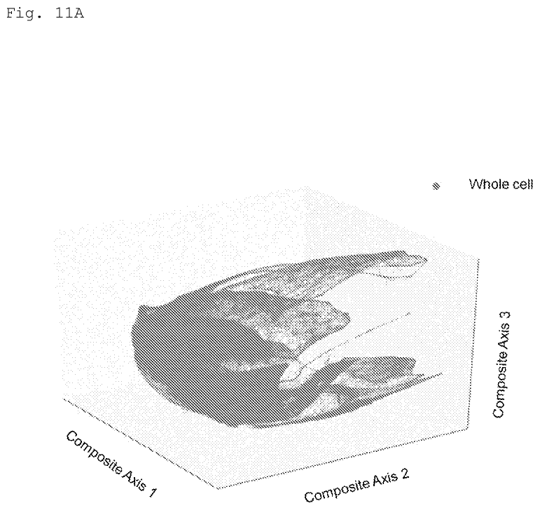

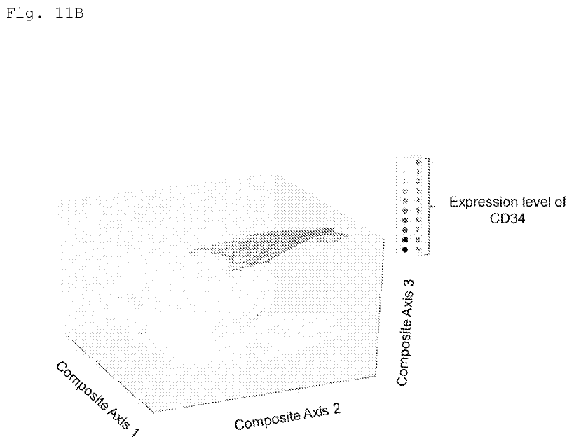

Quantitative physical property data on human umbilical cord blood was acquired with a flow cytometer using a set of commercially available markers (including CD34), and a cell arranged spatial structure was obtained by the cell stratifying process using the quantitative physical property data of the present invention. FIG. 11A shows an example in which the cell arranged space is shown in three dimensions.

In the cell arranged spatial structure, the expression level of CD34 known to be expressed in certain long-term hematopoietic stem cells was classified into stages 0 to 9, and the stages in the expression level of CD34 in each cell were mapped on the cell arranged spatial structure (FIG. 11B). Cells with high expression levels of CD34 are concentrated at one end of the cell arranged spatial structure, indicating that the cell arranged spatial structure obtained by the cell stratifying process using the quantitative physical property data reflects the cell lineage relationship.

### Example 3: Stratification of Head and Neck Squamous Cell Carcinoma Cells

(1) Identification of Cancer Cells

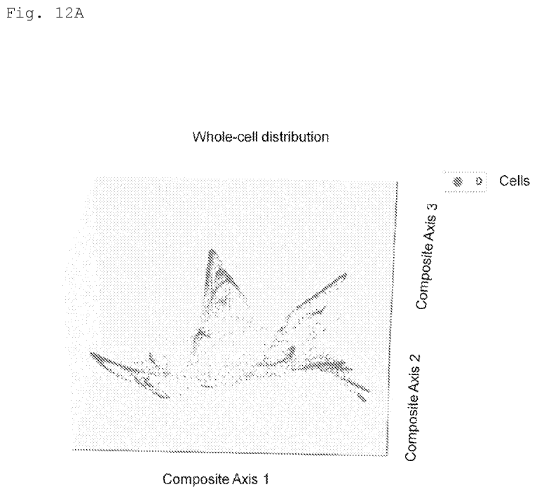

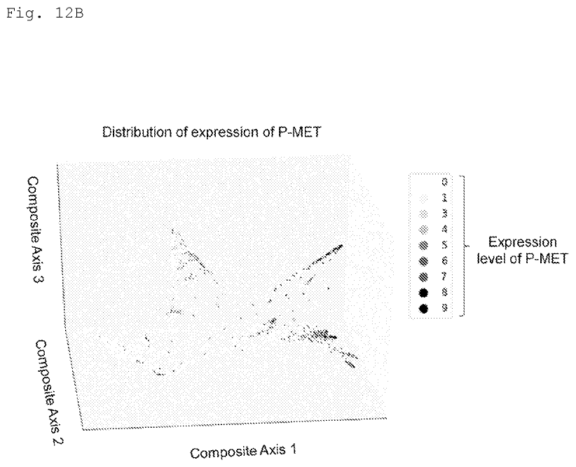

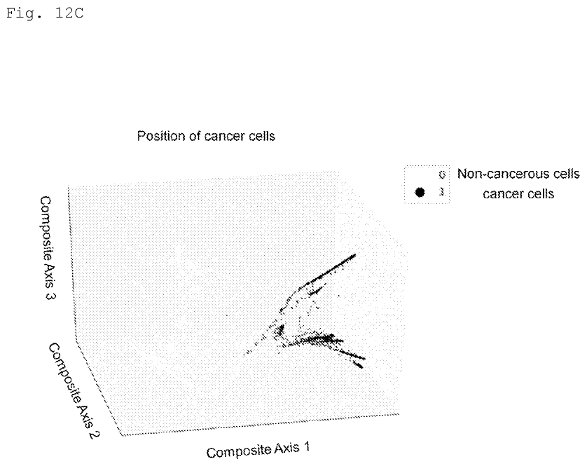

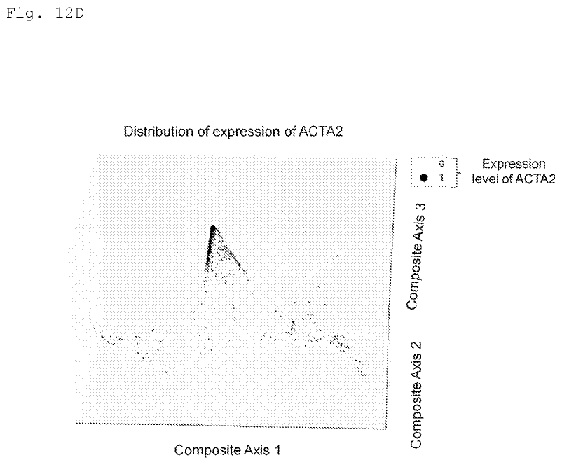

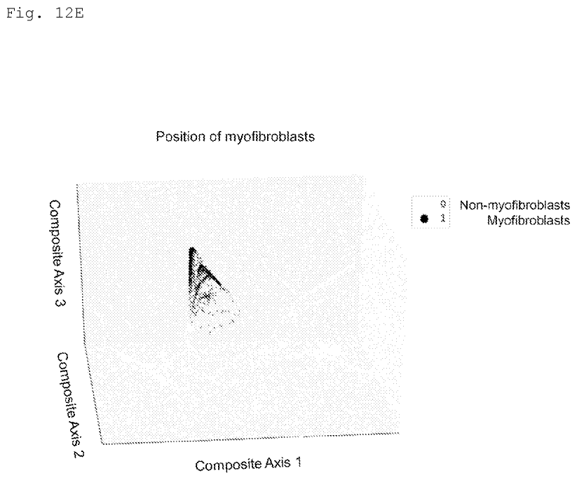

Concerning a biopsy sample containing cancer cells obtained from a head and neck squamous cell carcinoma patient by a known method (Cell, Vol. 171, no. 7, pp. 1611-1624), a cell arranged spatial structure was obtained by the cell stratifying process of the present invention using quantitative physical property data. FIG. 12A shows an example in which the cell arranged space is shown in three dimensions. Data obtained by identifying the expression level of P-MET known to be expressed in cancer cells by single-cell RNAseq was mapped on the cell arranged spatial structure to obtain FIG. 12B. The expression level of P-MET was classified into stages 0 to 9. Meanwhile, the positions of the cells determined to be cancer cells from the biological findings were mapped on the cell arranged spatial structure to obtain FIG. 12C.

(2) Identification of Muscle Fibroblasts

Data obtained by identifying the expression level of ACTA2 known to be expressed in muscle fibroblasts by single-cell RNAseq was mapped on the cell arranged spatial structure obtained in Example 3 to obtain FIG. 12D. The expression level of ACTA2 was classified into stages 0 to 1. Meanwhile, the positions of the cells determined to be muscle fibroblasts from the biological findings were mapped on the cell arranged spatial structure to obtain FIG. 12E.

From FIGS. 12A to E, it is understood that in the cell arranged spatial structure obtained by the cell stratification of the present invention, the region specified by the data indicating the biological properties and the position of the cells experimentally confirmed to have the biological properties show a high correlation.

### Example 4: Identification of Candidate Markers to Identify Human Long-Term Hematopoietic Stem Cells

In the cell arranged spatial structure obtained from the human umbilical cord blood obtained in Example 2, a combination of physical property data identifying the expression level distribution pattern of CD34 was identified, and the influence of each physical property data on the identification was quantified. More influential physical property data is classified as ++++ to + as important physical properties for identification of long-term hematopoietic stem cells and shown in the table below.

TABLE 1
Physical properties related to identification
of long-term hematopoietic stem cells

| Degree of | |
|---|---|
| importance | Physical properties |
| ++++ | CD217, CD191, CD328, CD34, GPR56, |
| | CD275, CD185, CD371, CD45RO, |
| | CD192, CD44, CD19, Notch 1, CD21, |
| | CD182, CD47, CD133, MRGX2, CD117, |
| | CD20, CD368, CD3, CD135, CD130, |
| | CD180, CD84, CD221, CD276, CD179b, |
| | CD85h, CD105, SUSD2, CD162, CD45RA, |
| | CD197, CD5, CXCR7, CD161, CD63, |
| | CD10, CCRL2, TRA 2 49, CD366, |
| | CD28, CD11c, CD106, CD85, CD87, |
| | CD11a, CD35, CD46, CD59, Integrin |
| | α9β1, CD99, SSEA-5, CD31, CD147, |
| | CD226, CD88, CD40, EGFR, CD131, |
| | CD58, CD126, SSEA-4, CD127/IL7R, |
| | CD194, CD43, Sialyl Lewis X, |
| | CD200R, TCR α/β, CD123, CD38 |
| ₊₊₊ | MSC, CD268, CD195, CD181, CD69, |
| | CD27, CD1b, CD18, KLRG1, CD24, |
| | CD97, CD328, CD79b, CD112, CD155, |
| | CD326, CD62L, CD45, TCR γ/δ, CD235ab, |
| | CD184, CD8a, CD52, CD49f, CD9, |
| | CD109, CD279, CD360, CD143, CD45RA, |
| | CD4, CD73, CD172a/b, CD154, CD370, |
| | Siglec-8, CD85k, CD170, Siglec-9, |
| | CD36, CD8a, CD218a, CD304, ROR1, |
| | CD1c, CD57, TNAP, CD114, TSLPR, |
| | CD278/ICOS, CD116/CSF2RA, CD103, |
| | Tim-4, TLT-2, HLA-E, CD317, CD2, |
| | CD71, CD269, CD148, HVEM, CD100, |
| | CD45RB, CD166, CD61, CD85d, CD298, |
| | CD82, DR3, CD164, CD263, CD215, |
| | CD132, CD158, MERTK |
| ++ | CD96, CD156c, CD230, CD66a/c/e, |
| | CD255, β2-microglobulin, CD26, |
| | Lymphotoxin β Receptor, MSC, NPC, |
| | CD32, CD81, CD201, CD274, CD48, |
| | CD183, CD243, TRA-2-54, MUC-13, |
| | CD111, CD223, CD22, CD179a, CD7, |
| | NKp80, CD334, CD199, CD300c, |
| | CD120b, CD360, TRA-1-81, CD102, |
| | CD11b/ITGAM, CD141, CD207, CD50, |
| | CD352, Delta Opioid Receptor, |
| | CD89, CD36L1, APCDD1, CD39, CD49e, |
| | CD1a, CD49b, CD30, CD324, |
| | CD122/1L2RB, CD267, CD90, CD335, |
| | CD294, CD196, HLA-A, B, C, CD303, |
| | CD200, CD229, CD95, CD213α1, CD13, |
| | CD62P, VEGFR-3, CD206, CD140a, |
| | TRA-1-60-R, CD92, CD64, Jagged 2, |
| | CCR10, CLEC4A, CD107a |
| + | FcεRIα, CD355, CD15, CD142, CD272, |
| | CD93, CD271, 4-1BB Ligand, CD158b, |
| | CD365, Ksp37, IL-28RA, CD85, CD94, |
| | CD369, CD74, CD115, CD42b, CD282, |
| | CD152, CD340, CD338, CD119, GARP, |
| | LOX-1, CD151, CD337, CD29, CD104, |
| | CD1d, CD55, GPR83, CXCL16, CD261, |
| | CD318, CD25, Notch 3, HLA-DR, CD85, |
| | TIGIT, CD124, CD6, CD172g, CD245, |
| | CD107b, CD258, CD165, CD262, CD336, |
| | erbB3, XCR1, CD38, CD138, CD70, |
| | CD16, CD144, CD213α2, CD344, CD323, |
| | Integrin β7, CD160, CD158e1, CD319, |
| | CD49c, CD354, CD314, IFN-γ R b chain, |
| | CD23, CD231, CD203c, CD140b, B7-H4, |
| | Notch 4 |

The physical properties can be used as candidate markers to identify long-term hematopoietic stem cells. Analyzing of the biological properties of the cells identified by the candidate markers makes it possible to identify candidate markers that identify long-term hematopoietic stem cells with higher accuracy. The degree of importance is given to the candidate markers using the cell stratification of the present invention at each analysis stage, so that it is possible to rapidly achieve high accuracy of the candidate markers.

### INDUSTRIAL APPLICABILITY

According to the present invention, the cell fraction arranged spatial structure and/or the cell arranged spatial structure obtained by stratifying cells is visualized by any method, so that it is possible to support the identification of cells at an initial stage of differentiation, i.e., cells close to stem cells, and the grasping of branched differentiation lineage relationship. Further, the utilization of the cell stratification of the present invention makes it possible to identify tissue stem cells and cancer stem cells inexpensively in a shorter period of time, and it is also possible to perform current and future profiling (such as a health or disease state) of each individual and to provide beneficial information for providing a personalized therapy. Further, the identification of the cell state of the individual and the relation between the medication history and/or the dietary constituent are investigated, so that it possible to support the screening of the personalized therapeutic agent and/or support the personalized instruction of medication and diet. Further, the cell stratification of the present invention is performed on a cell population obtained after culturing purified cells isolated from a population of cells once obtained from a living body for a certain period of time, thereby allowing for evaluation of the differentiation mode of the purified cells. Furthermore, the relation between the culture conditions when culturing the purified cells for a certain period of time and the evaluation of the differentiation mode is clarified, so that it is possible to support screening for optimal culture conditions for enriching specific differentiating cells from the purified cells. In addition, the utilization of the cell stratification of the present invention makes it possible to evaluate the cell content of specific cells contained in the cell population, the state and direction of differentiation in the cell population, and to evaluate the quality of the cells.

1. A method for identifying cells having a specific biological property,
the method comprising the steps of:
(1) stratifying each cell or cell fraction utilizing similarity of quantitative physical property data of each cell in a sample to obtain a cell fraction arranged spatial structure or a cell arranged spatial structure similar to a cell differentiation lineage; and
(2) identifying a region within the cell fraction arranged spatial structure or the cell arranged spatial structure utilizing biological property data of each cell.

2. The method according to claim 1, wherein the identification of the region utilizing the biological property data of each cell is identification by the amount or presence or absence of a marker associated with a biological property in each cell or cell fraction.

3. The method according to claim 1, wherein the identification of the region utilizing the biological property data of each cell is identification utilizing a position of cells having a marker associated with a biological property on the cell fraction arranged spatial structure or the cell arranged spatial structure.

4. A method for isolating cells using the method for identifying cells having a specific biological property according to claim 1,
the method comprising the steps of:
stratifying each cell or cell fraction as defined in step (1);
identifying a region within the cell fraction arranged spatial structure or the cell arranged spatial structure as defined in step (2); and
; and
further obtaining a gating parameter structure for separating the cells having a specific biological property.

5. A method for identifying a set of candidate markers for identifying cells having a specific biological property using the method for identifying cells having a specific biological property of claim 1,
the method comprising a step of
stratifying each cell or cell fraction as defined in step (1); and
identifying a region within the cell fraction arranged spatial structure or the cell arranged spatial structure as defined in step (2).

6. A marker for identifying cells having a specific biological property,
the marker comprising: a quantitative physical property selected from CD217, CD191, CD328, CD34, GPR56, CD275, CD185, CD371, CD45RO, CD192, CD44, CD19, Notch 1, CD21, CD182, CD47, CD133, MRGX2, CD117, CD20, CD368, CD3, CD135, CD130, CD180, CD84, CD221, CD276, CD179b, CD85h, CD105, SUSD2, CD162, CD45RA, CD197, CD5, CXCR7, CD161, CD63, CD10, CCRL2, TRA-2-49, CD366, CD28, CD11c, CD106, CD85, CD87, CD11a, CD35, CD46, CD59, Integrin α9β1, CD99, SSEA-5, CD31, CD147, CD226, CD88, CD40, EGFR, CD131, CD58, CD126, SSEA-4, CD127/IL7R, CD194, CD43, Sialyl Lewis X, CD200R, TCR α/β, CD123, CD38, MSC, CD268, CD195, CD181, CD69, CD27, CD1b, CD18, KLRG1, CD24, CD97, CD328, CD79b, CD112, CD155, CD326, CD62L, CD45, TCR γ/δ, CD235ab, CD184, CD8a, CD52, CD49f, CD9, CD109, CD279, CD360, CD143, CD45RA, CD4, CD73, CD172a/b, CD154, CD370, Siglec-8, CD85k, CD170, Siglec-9, CD36, CD8a, CD218a, CD304, ROR1, CD1c, CD57, TNAP, CD114, TSLPR, CD278/ICOS, CD116/CSF2RA, CD103, Tim-4, TLT-2, HLA-E, CD317, CD2, CD71, CD269, CD148, HVEM, CD100, CD45RB, CD166, CD61, CD85d, CD298, CD82, DR3, CD164, CD263, CD215, CD132, CD158, MERTK, CD96, CD156c, CD230, CD66a/c/e, CD255, β2-microglobulin, CD26, Lymphotoxin β Receptor, MSC, NPC, CD32, CD81, CD201, CD274, CD48, CD183, CD243, TRA-2-54, MUC-13, CD111, CD223, CD22, CD179a, CD7, NKp80, CD334, CD199, CD300c, CD120b, CD360, TRA-1-81, CD102, CD11b/ITGAM, CD141, CD207, CD50, CD352, Delta Opioid Receptor, CD89, CD36L1, APCDD1, CD39, CD49e, CD1a, CD49b, CD30, CD324, CD122/IL2RB, CD267, CD90, CD335, CD294, CD196, HLA-A, B, C, CD303, CD200, CD229, CD95, CD213α1, CD13, CD62P, VEGFR-3, CD206, CD140a, TRA-1-60-R, CD92, CD64, Jagged 2, CCR10, CLEC4A, CD107a, FcεRIα, CD355, CD15, CD142, CD272, CD93, CD271, 4-1BB Ligand, CD158b, CD365, Ksp37, IL-28RA, CD85, CD94, CD369, CD74, CD115, CD42b, CD282, CD152, CD340, CD338, CD119, GARP, LOX-1, CD151, CD337, CD29, CD104, CD1d, CD55, GPR83, CXCL16, CD261, CD318, CD25, Notch3, HLA-DR, CD85, TIGIT, CD124, CD6, CD172g, CD245, CD107b, CD258, CD165, CD262, CD336, erbB3, XCR1, CD38, CD138, CD70, CD16, CD144, CD213α2, CD344, CD323, Integrin β7, CD160, CD158e1, CD319, CD49c, CD354, CD314, IFN-γ R b chain, CD23, CD231, CD203c, CD140b, B7-H4, and Notch 4; or a combination of these quantitative physical properties, wherein the cells are hematopoietic stem cells.

7. A detection kit for identifying cells having a specific biological property,
the detection kit comprising a reagent for quantifying or detecting the marker as defined in claim 6 or a combination thereof, wherein the cells are hematopoietic stem cells.

8. A method for culturing cells using the method of identifying cells according to claim 1,
the method comprising the steps of:
stratifying each cell or cell fraction as defined in step (1);
identifying a region within the cell fraction arranged spatial structure or the cell arranged spatial structure as defined in step (2); and
; and
culturing a cell population under conditions where the identified cells are enriched.

9. A method for evaluating a content of cells having a specific biological property in a test sample,
the method comprising a step of comparing a cell fraction arranged spatial structure or a cell arranged spatial structure similar to a cell differentiation lineage obtained by stratifying each cell or cell fraction utilizing similarity of quantitative physical property data of each cell in a reference sample, with a cell fraction arranged spatial structure or a cell arranged spatial structure similar to a cell differentiation lineage obtained by stratifying each cell or cell fraction utilizing similarity of quantitative physical property data of each cell in a test sample.

10. A method for evaluating a differentiation state of cells in a test sample using the method for evaluating a content of cells as defined in claim 9,
the method comprising the step of comparing a cell fraction arranged spatial structure or a cell arranged spatial structure.

11. A method for evaluating a differentiation direction of cells in a test sample using the method for evaluating a content of cells as defined in claim 9,
the method comprising the step of comparing a cell fraction arranged spatial structure or a cell arranged spatial structure, under a plurality of conditions.

12. A method for evaluating a culture method using the method for evaluating a content of cells as defined in claim 9,
the method comprising the step of comparing a cell fraction arranged spatial structure or a cell arranged spatial structure, at each time point before and after culture under specific conditions.

13. A program for causing a computer to execute the method for identifying cells having a specific biological property according to claim 1, comprising the steps of:
(1) stratifying each cell or cell fraction as defined in step (1);
(2) identifying a region within the cell fraction arranged spatial structure or the cell arranged spatial structure as defined in step (2); and
(3) further identifying a position of cells having a specific biological property in the cell fraction arranged spatial structure or the cell arranged spatial structure.

14. A program for causing a computer to execute a step of visualizing biological property data obtained by testing cells whose position in a cell fraction arranged spatial structure or a cell arranged spatial structure is identified, wherein the cell fraction arranged spatial structure or the cell arranged spatial structure being similar to a cell differentiation lineage is obtained by stratifying each cell or cell fraction as defined in step (1) of claim 1.

15. A system comprising a computer,
wherein the computer includes:
(1) an input unit that inputs quantitative physical data and biological property data for each cell;
(2) a process unit that generates a cell fraction arranged spatial structure or a cell arranged spatial structure similar to a cell differentiation lineage by a stratifying process; and
(3) an output unit that outputs the cell fraction arranged spatial structure, the cell arranged spatial structure or a combination of physical properties of cells having a specific biological property.

16. (canceled)
